(19) United States
(12) Patent Application Publication
(10) Pub. No.: US 2007/0253979 A1
(43) Pub. Date: Nov. 1, 2007

(54) Immunogenic Hiv Compositions and Related Methods

(76) Inventor: Ronald B. Moss, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/570,177

(22) PCT Filed: Aug. 27, 2004
(86) PCT No.: PCT/US04/27995

§ 371 Date: Feb. 27, 2007

Related U.S. Application Data
(60) Provisional application No. 60/498,804, filed on Aug. 28, 2003.

(51) Int. Cl. A61K 39/00; C12N 7/00

(52) U.S. Cl. 424/208.1; 435/235.1

(57) ABSTRACT

The invention provides immunogenic compositions which enhance the duration and strength of the immune response in a mammal. The immunogenic compositions contain an HIV antigen, an immunomer and an adjuvant. The HIV antigen can be a whole-killed HIV virus devoid of outer envelope protein gp120. Alternatively, the HIV antigen can be a whole-killed HIV virus, or a p24 antigen. Also provided are kits, the components of which, when combined, produce the immunogenic compositions of the invention. The invention also provides methods of making the immunogenic compositions, by combining an HIV antigen, an immunomer and optionally an adjuvant. The invention further provides a method of immunizing a mammal, by enhancing an immune response in the mammal by administering to the mammal an immunogenic composition containing an HIV antigen, an immunomer and optionally an adjuvant. Also provided is a method of inhibiting in a mammal by administering to the mammal an immunogenic composition containing an HIV antigen, an immunomer and optionally an adjuvant.

### BACKGROUND INFORMATION

This invention relates to Acquired Immunodeficiency Syndrome (AIDS) and, more specifically, to immunogenic compositions for use in preventing and treating AIDS.

More than 30 million people world wide are now infected with the human immunodeficiency virus (HIV), the virus responsible for AIDS. About 90% of HIV infected individuals live in developing countries, including sub-Saharan Africa and parts of South-East Asia, although the HIV epidemic is rapidly spreading throughout the world. Anti-viral therapeutic drugs that reduce viral burden and slow the progression to AIDS have recently become available. However, these drugs are prohibitively expensive for use in developing nations. Thus, there remains an urgent need to develop effective preventative and therapeutic vaccines to curtail the global AIDS epidemic.

To date, HIV has proven a difficult target for effective vaccine development. Because of the propensity of HIV to rapidly mutate, there are now numerous strains predominating in different parts of the world whose epitopes differ. Additionally, in a particular infected individual, an HIV virus can escape from the control of the host immune system by developing mutations in an epitope. There remains a need to develop improved HIV vaccines that stimulate the immune system to recognize a broad spectrum of conserved epitopes, including epitopes from the p24 core antigen.

During the 1990's, more than 30 different candidate HIV-1 vaccines entered human clinical trials. These vaccines elicit various humoral and cellular immune responses, which differ in type and strength depending on the particular vaccine components. There remains a need to develop HIV vaccine compositions that strongly elicit the particular immune responses correlated with protection against HIV infection.

The nature of protective HIV immune responses has been addressed through studies of individuals who have remained uninfected despite repeated exposure to HIV, or who have been infected with HIV for many years without developing AIDS. These studies have shown that CD4+ T helper cells correlate well with protection against HIV infection and subsequent disease progression. Besides antigen-specific CD4+ helper T cell responses, CD8+ cytotoxic T cell responses are considered important in preventing initial HIV infection and disease progression. During an effective antiviral immune response, activated CD8+ T cells directly kill virus-infected cells and secrete cytokines with antiviral activity.

The β-chemokine system also appears to be important in protection against initial HIV infection and disease progression. Infection of immune cells by most primary isolates of HIV requires interaction of the virus with CCR5, whose normal biological role is as the principal receptor for the β-chemokines RANTES, MIP-1α and MIP-β. Genetic polymorphisms resulting in decreased expression of the CCR5 receptor have been shown to provide resistance to HIV infection. Additionally, a significant correlation between β-chemokine levels and resistance to HIV infection, both in exposed individuals and in cultured cells, has been demonstrated. It has been suggested that β-chemokines may block HIV infectivity by several mechanisms, including competing with or interfering with HIV binding to CCR5, and downregulating surface CCR5.

Because of the importance of β-chemokines in preventing initial HIV infection and disease progression, an effective HIV immunogenic composition should induce high levels of β-chemokine production, both prior to infection and in response to infectious virus. HIV immunogenic compositions capable of inducing β-chemokine production have been described. However, immunogenic compositions that stimulate high levels of β-chemokine production, induce strong, durable HIV-specific Th1 cellular and humoral immune response with HIV-specific cytotoxic activity have not been described.

Compositions that elicit certain types of HIV-specific immune responses may not elicit other important protective responses. For example, Dem1 et al., Clin. Chem. Lab. Med. 37:199-204 (1999), describes a vaccine containing an HIV-1 gp160 envelope antigen, an immunostimulatory DNA sequence and alum adjuvant, which, despite inducing an antigen-specific Th1-type cytokine response, was incapable of inducing an antigen-specific cytotoxic T lymphocyte response. Furthermore, a vaccine containing only envelope antigens would not be expected to induce an immune response against the more highly conserved core proteins of HIV.

Thus, there exists a need for immunogenic compositions and methods that will either help to prevent HIV infection or at least slow progression to AIDS in infected individuals. The present invention satisfies this need and provides related advantages as well.

### SUMMARY OF THE INVENTION

The invention provides immunogenic compositions which can be used to enhance the potency of immune responses in a mammal. The immunogenic compositions of the invention can enhance the breadth, type, strength and duration of the immune responses induced. The immunogenic compositions contain an optimized HIV antigen, an isolated nucleic acid molecule containing an immunomer and optionally an adjuvant. The HIV antigen can be a whole-killed HIV virus devoid of outer envelope protein gp120. The HIV antigen can also be protease-defective HIV particles such as L2 particles. Alternatively, the HIV antigen can be a whole-killed HIV virus, or a combination of selected HIV antigens or peptides, including p24 antigen, nef, gp41, and the like.

In the immunogenic compositions of the invention in which an adjuvant is present, the adjuvant can be suitable for administration to a human. An exemplary adjuvant is Incomplete Freund's Adjuvant.

The immunogenic compositions of the invention can further enhance β-chemokine levels, interferon-γ (IFNγ), interleukin 2 (IL2), tumor necrosis factor alpha (TNFα), and interleukin 15 (IL15) production, and/or HIV-specific IgG2b antibody production in a mammal. The immunogenic compositions of the invention can also enhance HIV specific helper CD4+ T cells, an HIV-specific cytotoxic T lymphocyte response, and non cytotoxic suppressive T lymphocyte responses in a mammal.

Also provided are kits, which contain an HIV antigen, an immunomer and optionally an adjuvant. The components of the kits, when combined, produce the immunogenic compositions of the invention.

The invention also provides methods of making the immunogenic compositions, by combining an HIV antigen, an immunomer and optionally an adjuvant. The components can be combined ex vivo or in vivo to arrive at the immunogenic compositions.

The invention also provides a method of immunizing a mammal by administering to the mammal an immunogenic composition containing an HIV antigen, an isolated nucleic acid molecule containing immunomer and optionally an adjuvant. Also provided is a method of inhibiting AIDS, by enhancing an immune response in the mammal by administering to the mammal an immunogenic composition containing an HIV antigen, an isolated nucleic acid molecule containing an immunomer and optionally an adjuvant. In the methods of the invention, the mammal can be a primate, such as a human, or a rodent. In certain embodiments of the method, the primate is a pregnant mother or an infant. A human can be HIV seronegative or HIV seropositive. The immunogenic compositions can advantageously be administered to the mammal two or more times and by a variety of administration routes, including subcutaneously, intramuscularly and intramucosally.

### BRIEF DESCRIPTION OF THE DRAWINGS

FIG. 1 shows the chemical structures of exemplary linkers for linking oligonucleotides to form an immunomer (Yu et al., J. Med. Chem. 45:4540-4548 (2002); Yu et al., Nucl. Acids Res. 30:4460-4469 (2002)).

FIG. 2 shows a schematic diagram of the immunomer HYB2055, also known as Amplivax™.

FIG. 3 shows the induction of HIV-specific cytokines RANTES, MIP1α, MIP1β, interleukin-10 (IL-10) and IL-5 by HIV-1 Immunogen. The immunogen was administered subcutaneously. “*” indicates significance vs. saline.

FIG. 4 shows HIV-1 immunogen induced production of HIV-specific interferon-γ (IFNγ) is enhanced by Amplivax™ in a dose dependent manner. “*” indicates significance vs. HIV-1 immunogen alone.

FIG. 5 shows the effect of Amplivax™ on production levels of RANTES, MIP1α, MIP1β, IL-1 and IL-5. The immunogen was administered subcutaneously. “*” indicates significance vs. saline.

FIG. 6 shows enhanced HIV-specific IFNγ production by Amplivax™. Similar results were found for RANTES, MIP1α, MIP1β and IL-10. The immunogen was administered subcutaneously. “*” indicates significance vs. saline.

FIG. 7 shows the enhancing effect of Amplivax™ on HIV-specific IFNγ-secreting T cells in an Elispot assay. Immunogen was administered subcutaneously. “*” indicates significance vs. HIV-1 immunogen alone.

FIG. 8 shows that HIV-specific IFNγ production was enhanced by Amplivax™ in a dose dependent manner. Immunogen was administered subcutaneously. “*” indicates significance vs. HIV-1 immunogen alone.

FIG. 9 shows that HIV-specific RANTES production was enhanced by Amplivax™ in a dose dependent manner. Immunogen was administered subcutaneously. “*” indicates significance vs. HIV-1 immunogen alone.

FIG. 10 shows that HIV-specific MIP-1α production was enhanced by Amplivax™ in a dose dependent manner. Immunogen was administered subcutaneously. “*” indicates significance vs. HIV-1 immunogen alone.

FIG. 11 shows that HIV-specific MIP-1β production was enhanced by Amplivax™ in a dose dependent manner. Immunogen was administered subcutaneously. “*” indicates significance vs. HIV-1 immunogen alone.

FIG. 12 shows that HIV-specific Il-10 production was enhanced by Amplivax™ in a dose dependent manner. Immunogen was administered subcutaneously. “*” indicates significance vs. HIV-1 immunogen alone

FIG. 13 shows that HIV-specific IL-5 production was reduced by Amplivax™ given subcutaneously (SC). “*” indicates significance vs. HIV-1 immunogen alone.

FIG. 14 shows the effect of Amplivax™ on HIV-1 immunogen-induced p24 antibody titers in mice. Immunogen was administered subcutaneously.

FIG. 15 shows that HIV-1 whole killed vaccine in IFA (HIV-1 immunogen) induced HIV specific cytokine production upon subcutaneous (SC) and intramuscular (IM) administration.

FIG. 16 shows that Amplivax™ can be added pre- or post-emulsion with IFA and enhance IFNγ production.

FIG. 17 shows that Amplivax™ can be added pre- or post-emulsion with IFA and enhance RANTES production.

FIG. 18 shows that HIV-1 whole killed vaccine antigen with Amplivax™ triggered HIV-specific IFNγ production in mice immunized subcutaneously without IFA. “*” indicates significance vs. HIV-1 immunogen (IM). HIV antigen is HIV whole killed vaccine without IFA.

FIG. 19 shows that HIV-1 whole killed vaccine antigen with Amplivax™ triggered HW-specific IFNγ-secreting CD8+ T cell activity in mice immunized subcutaneously without IFA. “*” indicates significance vs. HIV-1 immunogen (administered IM). HIV antigen is HIV whole killed vaccine without IFA.

FIG. 20 shows that HIV-1 whole killed vaccine antigen with Amplivax™ triggered HIV-specific RANTES production in mice immunized subcutaneously without IFA. “*” indicates significance vs. HIV-1 immunogen (administered IM). HIV antigen is HIV whole killed vaccine without IFA.

FIG. 21 shows that percentages of α-defensin producing CD8+ T cells are increased by Amplivax™ added ex vivo.

FIG. 22 shows HIV-specific IFNγ-producing CD8+ T cells in REMUNE® treated patients and HIV positive controls (0 μg/ml Amplivax™).

FIG. 23 shows HIV-specific IFNγ-producing CD8+ T cells in the presence of 0.1 μg/ml of Amplivax™ added ex vivo.

FIG. 24 shows HIV-specific IFNγ-producing CD8+ T cells in the presence of 1 μg/ml of Amplivax™ added ex vivo.

FIG. 25 shows HIV-specific IFNγ-producing CD8+ T cells in the presence of 10 μg/ml of Amplivax™ added ex vivo.

FIG. 26 shows IFN-γ ELIspot assay in peripheral blood mononuclear cells (PBMCs). HYB2055 was used at 1 μg/ml.

FIG. 27 shows phenotypic changes in CD4 T cells post 1st injection of REMUNE® in antiretroviral therapy (ART) naive patients.

FIG. 28 shows phenotypic changes in CD8 T cells post 1st injection of REMUNE® in ART naive patients.

### DETAILED DESCRIPTION OF THE INVENTION

The present invention provides immunogenic HIV compositions containing an HIV antigen, an isolated nucleic acid molecule containing an immunomer, and optionally an adjuvant. Also provided are kits containing the components of such compositions, for use together. The invention also provides methods of immunizing a mammal with such compositions, or with the components of such compositions, so as to enhance the immune response in the immunized mammal relative to HIV antigen alone. Advantageously, the compositions of the invention can also induce HIV specific CD4 T helper cells and CD8+ T cells yielding potent Th1 immune responses against a broad spectrum of HIV epitopes, providing a strong HIV-specific cytotoxic T lymphocyte response. Thus, the immunogenic compositions of the invention are useful for preventing HIV infection and/or slowing progression to AIDS in infected individuals. The compositions and methods can be used to elicit potent Th1 cellular and humoral immune responses specific for conserved HIV epitopes, elicit HIV-specific CD4 T helper cells, HIV-specific cytotoxic T lymphocyte activity, stimulate production of chemokines and cyotokines such as chemokines, interferon-γ, interleukin 2 (IL2), interleukin 7 (IL7), interleukin 15 (IL15), alpha-defensin, and the like, and increase memory cells. Such vaccines can be administered via various routes of administration. Such vaccines can be used to prevent maternal transmission of HIV, for vaccination of newborns, children and high-risk individuals, and for vaccination of infected individuals. Such vaccines can also be used in combination with other HIV therapies, including antiretroviral therapy (ART) with various combinations of nuclease and protease inhibitors and agents to block viral entry, such as T20 (see Baldwin et al., Curr. Med. Chem. 10:1633-1642 (2003)).

As used herein, the term “HIV” refers to all forms, subtypes and variations of the HIV virus, and is synonymous with the older terms HTLVIII and LAV. Various cell lines capable of propagating HIV or permanently infected with the HIV virus have been developed and deposited with the ATCC, including HuT 78 cells and the HuT 78 derivative H9, as well as those having accession numbers CCL 214, TIB 161, CRL 1552 and CRL 8543, which are described in U.S. Pat. No. 4,725,669 and Gallo, Scientific American 256:46 (1987).

As used herein, the term “whole-killed HIV virus” refers to an intact, inactivated HIV virus. An inactivated HIV refers to a virus that cannot infect and/or replicate.

As used herein, the term “outer envelope protein” refers to that portion of the 5 membrane glycoprotein of a retrovirus which protrudes beyond the membrane, as opposed to the transmembrane protein, gp41.

As used herein, the term “HIV virus devoid of outer envelope proteins” refers to a preparation of HIV particles or HIV gene products devoid of the outer envelope protein gp120, but contains the more genetically conserved parts of the virus (for example, p24 and gp41). An HIV devoid of the outer envelope protein gp120 is also referred to herein as REMUNE™.

As used herein, the term “HIV p24 antigen” refers to the gene product of the gag region of HIV, characterized as having an apparent relative molecular weight of about 24,000 daltons designated p24. The term “HIV p24 antigen” also refers to modifications and fragments of p24 having the immunological activity of p24. Those skilled in the art can determine appropriate modifications of p24, such as additions, deletions or substitutions of natural amino acids or amino acid analogs, that serve, for example, to increase its stability or bioavailability or facilitate its purification, without destroying its immunological activity. Likewise, those skilled in the art can determine appropriate fragments of p24 having the immunological activity of p24. An immunologically active fragment of p24 can have from 6 residues from the polypeptide up to the full length polypeptide minus one amino acid. Other HIV antigens encoded by other HIV gene products can include fragments or modifications similar to those described above for the HIV p24 antigen. Other exemplary HIV antigens include, for example, gp41, nef, and the like.

As used herein, an “immunomer” refers to an oligonucleotide comprising two smaller oligonucleotides linked at their 3′ ends, resulting in an oligonucleotide having two 5′ ends. The two smaller oligonucleotides of the immunomer can be identical or non-identical sequences and/or lengths, but generally are identical. In addition to its immunostimulatory activity, an immunomer contains a 3′-3′ linkage and therefore has no free 3′ end, thus increasing resistance to nuclease digestion. The smaller oligonucleotides of the immunomer are generally at least about 5 or 6 nucleotides that are linked together to form two 5′ ends, but can be longer such as 7, 8, 9, 10, 11, 12, 13, 14, 15, 16, 17, 18, 19, 20, or even longer smaller oligonucleotides of the immunomer. One skilled in the art can readily determine a length and/or sequence of immunomer sufficient to stimulate an immune response greater than that seen with antigen alone. Thus, in one embodiment, an immunomer comprises two identical oligonucleotides linked via their 3′ ends. An immunomer can also include modified bases. Immunomers are described, for example, in Kandimalla et al., Bioorg. Med. Chem. 9:807-813 (2001); Yu et al., Nucl. Acids Res. 30:4460-4469 (2002); Yu et al., Bioorg. Med. Chem. 11:459-464 (2003); Bhagat et al., Biochem. Biophys. Res. Comm. 300:853-861 (2003); and Yu et al., Biochem. Biophys. Res. Comm. 297:83-90 (2002); Yu et al., Nucl. Acids Res. 30:1613-1619 (2002); Yu et al., J. Med. Chem. 45:4540-4548 (2002); Kandimalla et al., Bioconjugate Chem. 13:966-974 (2002); Yu et al., Bioorganic Med. Chem. Lett. 10:2585-2588 (2000); Agrawal and Kandimalla, Trends Mol. Med. 8:114-121 (2002); each of which is incorporated herein by reference. Such immunomers can have more potent immunostimulatory activity than immunostimulatory sequences containing CpG. An immunomer enhances the immune response in a mammal when administered in combination with an antigen. An immunomer can be a CpG immunomer or CpG-free immunomer, as discussed below. An exemplary immunomer is described in Examples X and XI.

As used herein, a “CpG immunomer” refers to an immunomer, as described above, that specifically contains a CpG motif. Thus, a CpG immunomer is an oligonucleotide comprising two identical or non-identical smaller oligonucleotides, where at least one of the smaller oligonucleotides contains at least one CpG motif.

As used herein, a “CpG-free immunomer” refers to an immunomer that specifically excludes a CpG motif. Thus, a CpG-free immunomer is an oligonucleotide comprising two identical or non-identical smaller oligonucleotides, where neither of the smaller oligonucleotides contains a CpG motif.

An immunomer can contain modified bases (see Kandimalla et al., supra, 2001). For example, an immunomer can contain analogues of CpG. For example, an immunomer can contain a pyrimidine analog of deoxycytosine, designated Y. Particularly useful deoxycytidine analogs for use in an immunomer are deoxy-5-hydroxycytidine or deoxy-N4-ethylcytidine. In another embodiment, an immunomer can contain a purine analog of guanine, designated R. A particularly useful deoxyguanine analog is 7-deazguanine. Thus, an immunomer can contain a YpG motif, a CpR motif, or a YpR motif, where Y and R are analogs of cytosine and guanine, respectively.

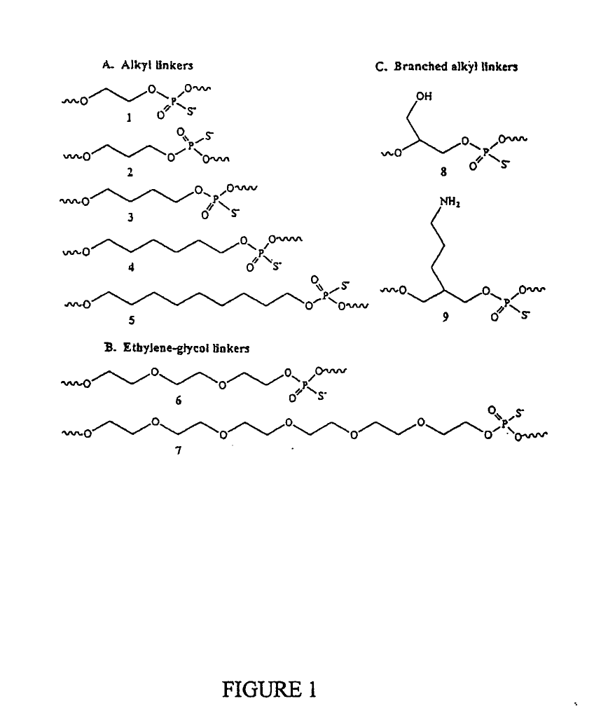

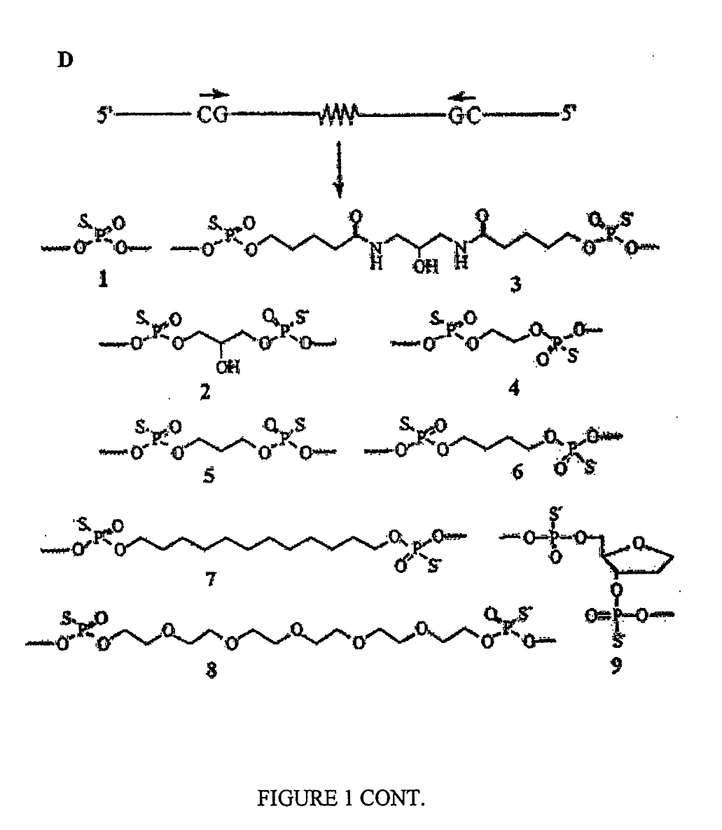

Methods of linking two smaller oligonucleotides to form an immunomer have been described previously (Kandimalla et al., Bioorg. Med. Chem. 9:807-813 (2001); Yu et al., Nucl. Acids Res. 30:4460-4469 (2002); Yu et al., Bioorg. Med. Chem. 11:459-464 (2003); Bhagat et al., Biochem. Biophys. Res. Comm. 300:853-861 (2003); and Yu et al., Biochem. Biophys. Res. Comm. 297:83-90 (2002); Yu et al., Nucl. Acids Res. 30:1613-1619 (2002); Yu et al., J. Med. Chem. 45:4540-4548 (2002); Kandimalla et al., Bioconjugate Chem. 13:966-974 (2002); Yu et al., Bioorganic Med. Chem. Lett. 10:2585-2588 (2000); Agrawal and Kandimalla, Trends Mol. Med. 8:114-121 (2002)). Exemplary linkers include, for example, 3′-3′ linkages via a glyceryl linker (Yu et al., Biochem. Biophys. Res. Comm. 297:83-90 (2002). Linkers can be alkyl, branched alkyl or ethylene-glycol linkers, as described in Yu et al., J. Med. Chem. 45:4540-4548 (2002)(see FIG. 1). One skilled in the art will readily recognize that these and other methods can be used to link oligonucleotides via their 3′ ends to generate two free 5′ ends.

As used herein, the term “immunostimulatory sequence” or “ISS” refers to a nucleotide sequence containing an unmethylated CpG motif that is capable of enhancing the immune response in a mammal when administered in combination with an antigen. Immunostimulatory sequences are described, for example, in PCT publication WO 98/55495.

As used herein, the term “nucleic acid molecule containing an immunomer” refers to a linear, circular or branched single- or double-stranded DNA or RNA nucleic acid that contains an immunomer. A nucleic acid molecule containing an immunomer can contain a single immunomer. A nucleic acid molecule can also contain more than one immunomer if one or both of the free 5′ ends is linked to the 5′ end of another nucleic acid sequence to generate another potential 3′ end for linkage of an additional immunomer. Such a nucleic acid molecule, in addition to the immunomer, can be of any length greater than 6 bases or base pairs, and is generally greater than about 15 bases or base pairs, such as greater than about 20 bases or base pairs, and can be several kb in length. When such a nucleic acid containing an immunomer is circular, or when it contains multiple immunomers requiring 5′-5′ linkages, it is understood that the free 5′ ends are linked, for example, as described in Kandimalla et al., Bioconjugate Chem. 13:966-974 (2002), and Yu et al., Bioorgan. Med. Chem. 10:2585-2588 (2000), so long as the 5′-5′ linkage does not interfere with the immunostimulatory activity of the immunomer embedded in the nucleic acid, as is found with short immunomers (Kandimalla et al., supra, 2002, and Yu et al., supra, 2000). A nucleic acid containing an immunomer can additionally contain nucleic acid sequence encoding one or more HIV antigens for use as a DNA vaccine.

An immunomer or nucleic acid molecule containing an immunomer can be generated, for example, by chemically synthesizing oligonucleotides and chemically linking the oligonucleotides via their 3′ ends, as disclosed herein. In addition, an immunomer or nucleic acid molecule containing an immunomer can be generated by recombinantly synthesizing the two halves of the immunomer or nucleic acid containing an immunomer and chemically linking the two halves via their 3′ ends.

An immunomer can contain either natural or modified nucleotides or natural or unnatural nucleotide linkages. Modifications known in the art, include, for example, modifications of the 3′OH or 5′OH group, modifications of the nucleotide base, modifications of the sugar component, and modifications of the phosphate group. An unnatural nucleotide linkage can be, for example, a phosphorothioate linkage in place of a phosphodiester linkage, which increases the resistance of the nucleic acid molecule to nuclease degradation. Various modifications and linkages are described, for example, in PCT publication WVO 98/55495.

As used herein, the term “adjuvant” refers to a substance which, when added to an immunogenic agent, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as polysytrene, starch, polyphosphazene and polylactide/polyglycosides. Adjuvants can also include, for example, squalene mixtures (SAF-I), muramyl peptide, saponin derivatives, mycobacterium cell wail preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant (both complete and incomplete) can be used. In humans, Incomplete Freund's Adjuvant (IFA) is a particularly useful adjuvant. Various appropriate adjuvants are well known in the art and are reviewed, for example, by Warren and Chedid, CRC Critical Reviews in Immunology 8:83 (1988).

As used herein, “AIDS” refers to the symptomatic phase of HIV infection, and includes both Acquired Immune Deficiency Syndrome (commonly known as AIDS) and “ARC,” or AIDS-Related Complex, as described by Adler, Brit. Med. J. 294: 1145 (1987). The immunological and clinical manifestations of AIDS are well known in the art and include, for example, opportunistic infections and cancers resulting from immune deficiency.

As used herein, the term “inhibiting AIDS” refers to a beneficial prophylactic or therapeutic effect of the immunogenic composition in relation to HIV infection or AIDS symptoms. Such beneficial effects include, for example, preventing or delaying initial infection of an individual exposed to HIV; reducing viral burden in an individual infected with HIV; prolonging the asymptomatic phase of HIV infection; maintaining low viral loads in HIV infected patients whose virus levels have been lowered via anti-retroviral therapy (ART); increasing levels of CD4 T cells or lessening the decrease in CD4 T cells, both HIV-1 specific and non-specific, in drug naive patients and in patients treated with ART, increasing overall health or quality of life in an individual with AIDS; and prolonging life expectancy of an individual with AIDS. A clinician can compare the effect of immunization with the patient's condition prior to treatment, or with the expected condition of an untreated patient, to determine whether the treatment is effective in inhibiting AIDS.

As used herein, the term “enhances,” with respect to an immune response is intended to mean that the immunogenic composition elicits a greater immune response than does a composition containing HIV antigen alone. In the case where the immunogenic composition contains the three components HIV antigen, immunomer and adjuvant, the immunogenic composition elicits a greater immune response than does a composition containing any two of the three components of the immunogenic composition, administered in the same amounts and following the same immunization schedule. The components of the immunogenic compositions of the invention can act in synergy. An enhanced immune response can be, for example, increased production of chemokines and/or cytokines that promote memory cells, an increase in memory cells, an increase in IgG2b production, in increase in cytotoxic T lymphocyte activity, an increase in β-chemokine or IL15 production, and the like. As an example of an enhanced immune response, the immunogenic compositions of the invention can increase production of γ-interferon by both CD4 cells (helper function) and CD8 cells (cytotoxic T lymphocytes; CTLs).

As used herein, the term “β-chemokine” refers to a member of a class of small, chemoattractive polypeptides that includes RANTES, macrophage inflammatory protein-1β (MIP-1β) and macrophage inflammatory protein-1α (MIP-1α). The physical and functional properties of β-chemokines are well known in the art.

In the case of enhanced β-chemokine production, the β-chemokine production can be “HIV-specific β-chemokine production,” which refers to production of a β-chemokine in response to stimulation of T cells with an HIV antigen. Alternatively, or additionally, the β-chemokine production that is enhanced can be “non-specific β-chemokine production,” which refers to production of a β-chemokine in the absence of stimulation of cells with an HIV antigen.

As used herein, the term “kit” refers to components packaged or marked for use together. For example, a kit can contain an HIV antigen, an immunomer and an adjuvant in three separate containers. Alternatively, a kit can contain any two components in one container, and a third component and any additional components in one or more separate containers. Optionally, a kit further contains instructions for combining the components so as to formulate an immunogenic composition suitable for administration to a mammal.

The invention provides an immunogenic composition containing an HIV antigen, an immunomer, and optionally an adjuvant. The immunogenic composition enhances the immune response in a mammal administered the composition. In one embodiment, the immunogenic composition enhances an HIV-specific cytotoxic T lymphocyte (CTL) response in a mammal. In another embodiment, the immunogenic composition enhances HIV-specific CD4+ helper T cells.

In one embodiment, the HIV antigen in the immunogenic composition is a whole-killed HIV virus, which can be prepared by methods known in the art. For example, HIV virus can be prepared by culture from a specimen of peripheral blood of infected individuals. In an exemplary method of culturing HIV virus, mononuclear cells from peripheral blood (for example, lymphocytes) can be obtained by layering a specimen of heparinized venous blood over a Ficoll-Hypaque density gradient and centrifuging the specimen. The mononuclear cells are then collected, activated, as with phytohemagglutinin for two to three days, and cultured in an appropriate medium, preferably supplemented with interleukin 2 (IL2). The virus can be detected either by an assay for reverse transcriptase, by an antigen capture assay for p24, by immunofluorescence or by electron microscopy to detect the presence of viral particles in cells, all of which are methods well known to those skilled in the art.

Methods for isolating whole-killed HIV particles are described, for example, in Richieri et al., Vaccine 16:119-129 (1998), and U.S. Pat. Nos. 5,661,023 and 5,256,767. In one embodiment, the HIV virus is an HZ321 isolate from an individual infected in Zaire in 1976, which is described in Choi et al., AIDS Res. Hum. Retroviruses 13:357-361 (1997).

Various methods are known in the art for rendering a virus non-infectious (see, for example Hanson, MEDICAL VIROLOGY II (1983), de la Maza and Peterson, eds., Elsevier,). For example, the virus can be inactivated by treatment with chemicals or by physical conditions such as heat or irradiation. Preferably, the virus is treated with an agent or agents that maintain the immunogenic properties of the virus. For example, the virus can be treated with beta-propiolactone or gamma radiation, or both beta-propiolactone and gamma radiation, at dosages and for times sufficient to inactivate the virus.

In another embodiment, the HIV antigen in the immunogenic composition is a whole-killed HIV virus devoid of outer envelope proteins, which can be prepared by methods known in the art. In order to prepare whole-killed virus devoid of outer envelope proteins, the isolated virus is treated so as to remove the outer envelope proteins. Such removal is preferably accomplished by repeated freezing and thawing of the virus in conjunction with physical methods which cause the swelling and contraction of the viral particles, although other physical or non-physical methods, such as sonication, can also be employed alone or in combination.

In yet another embodiment, the HIV antigen in the immunogenic composition is one or more substantially purified gene products of HIV. Such gene products include those products encoded by the gag genes (p55, p39, p24, p17 and pl5), the pol genes (p66/p51 and p31-34) and the transmembrane glycoprotein gp41; and the nef protein. These gene products may be used alone or in combination with other HIV antigens. The HIV antigen can also be peptide fragments of HIV gene products that illicit an immune response.

The substantially purified gene product of HIV can be a substantially purified HIV p24 antigen or other HIVT antigens and gene products. p24, as well as other HIV antigens, can be substantially purified from the virus by biochemical methods known in the art, or can be produced by cloning and expressing the appropriate gene in a host organism such as bacterial, fungal or mammalian cells, by methods well known in the art. Alternatively, p24 antigen, or a modification or fragment thereof that retains the immunological activity of p24, as well as other HIV antigens or modifications or fragments thereof, can be synthesized, using methods well known in the art, such as automated peptide synthesis. Determination of whether a modification or fragment of p24 retains the immunological activity of p24, or other viral antigens retain their respective immunological activity, can be made, for example, by their ability to stimulate proliferation in vitro of previously immunized PBMCs as analyzed by conventional lymphocyte proliferation assays (LPA) known in the art (see Example III), by immunizing a mammal and comparing the immune responses so generated, or testing the ability of the modification or fragment to compete with p24 for binding to a p24 antibody, or other HIV antigens to their respective antibodies.

In still another embodiment, the HIV antigen in the immunogenic composition is a substantially purified gene product of a protease defective HIV (see U.S. Pat. Nos. 6,328,976 and 6,557,296).

The replication process for HIV-1 has an error rate of about one per 5-10 base pairs. Since the entire viral genome is just under 10,000 base pairs, this results in an error rate of about on base pair per replication cycle. This high mutation rate contributes to extensive variability of the viruses inside any one person and an even wider variability across populations.

This variability has resulted in three HIV-1 variants being described and around 10 subspecies of virus called “clades.” These distinctions are based on the structure of the envelope proteins, which are especially variable. The M (for major) variant is by far the most prevalent world wide. Within the M variant are clades A, B, C, D, E, F, G H, I, J and K, with clades A through E representing the vast majority of infections globally. Clades A, C and D are dominant in Africa. Clade B is the most prevalent in Europe, North and South America and Southeast Asia. Clades E and C are dominant in Asia. These clades differ from on another by as much as 35%.

There are two important results from the very high mutation rate of HIV-1 that have profound consequences for the epidemic. First, the high mutation rate is one of the mechanisms that allows the virus to escape from control by drug therapies. These new viruses represent resistant strains. The high mutation rate also allows the virus to escape the patient's immune system by altering the structures that are recognized by immune components. An added consequence of this extensive variability is that the virus can also escape from control by vaccines, and vaccines based on envelope proteins will likely be non-effective.

The greatest variation in structure is seen in the envelope proteins gp120 and gp41. Less variation is seen in the various internal proteins. As disclosed herein, REMUNE is an immunogen that is made from the whole virus without its gp120 proteins but contains most of the highly conserved epitopes of the HIV-1 virus. Both the number of these epitopes and their lower incidence of mutation mean that an HIV virus devoid of outer envelope proteins such as REMUNE stimulates the immune responses that have a greater chance of success within individuals. In addition, the HuT 78 cell line was purposely infected with a very early strain of HIV virus containing both clades A and G for conserved antigens, which have been retained across most variations in clades seen worldwide, and this HuT 78 HIV infected cell line provided virus used as HIV antigen. Thus, the use of an HIV virus with multiple early clades that is also devoid of outer envelope proteins for immunization can be effective across clades by providing conserved antigens that can be recognized by most patients.

The HIV antigen and an immunomer can be mixed together, or can be conjugated by either a covalent or non-covalent linkage. Methods of conjugating antigens and nucleic acid molecules are known in the art, and exemplary methods are described in PCT publication WO 98/55495.

An oliognucleotide component of an immunomer can be prepared using methods well known in the art including, for example, oligonucleotide synthesis, PCR, enzymatic or chemical degradation of larger nucleic acid molecules, and conventional polynucleotide isolation procedures. Methods of producing an oligonucleotide component of an immunomer, including an oligonucleotide containing one or more modified bases or linkages, are described, for example, in PCT publication WO 98/55495.

Those skilled in the art can readily determine whether a particular immunomer is effective in enhancing a desired immune response in a particular mammal by immunizing a mammal of the same species, or a species known in the art to exhibit similar immune responses, with a composition containing a particular immunomer. A variety of assays known in the art can then be used to characterize and compare the characteristics of the immune responses induced. For example, an optimized immunomer to include in an immunogenic composition for administration to a human can be determined in either a human or a non-human primate, such as a baboon, chimpanzee, macaque or monkey by evaluating its immune activity, for example, by LPA, ELISPOT, and/or ratios of IgG1/G2 antibody produced.

The immunogenic compositions of the invention can further contain an adjuvant, such as an adjuvant demonstrated to be safe in humans. An exemplary adjuvant is Incomplete Freund's Adjuvant (IFA). Another exemplary adjuvant contains mycobacterium cell wall components and monophosphoryl lipid A, such as the commercially available adjuvant DETOXT. Another exemplary adjuvant is alum. The preparation and formulation of adjuvants in immunogenic compositions are well known in the art.

Optionally, the immunogenic compositions of the invention can contain or be formulated together with other pharmaceutically acceptable ingredients, including sterile water or physiologically buffered saline. A pharmaceutically acceptable ingredient can be any compound that acts, for example, to stabilize, solubilize, emulsify, buffer or maintain sterility of the immunogenic composition, which is compatible with administration to a mammal and does not render the immunogenic composition ineffective for its intended purpose. Such ingredients and their uses are well known in the art.

The invention also provides kits containing an HIV antigen, immunomer, and optionally an adjuvant. The components of the kit, when combined, produce an immunogenic composition which enhances an immune response in a mammal.

The components of the kit can he combined ex vivo to produce an immunogenic composition containing an HIV antigen, an immunomer and optionally an adjuvant. Alternatively, any two components can be combined ex vivo, and administered with a third component, such that an immunogenic composition forms in vivo. For example, an HIV antigen can be emulsified in, dissolved in, mixed with, or adsorbed to an adjuvant and injected into a mammal, preceded or followed by injection of immunomer. Likewise, each component of the kit can be administered separately. Those skilled in the art understand that there are various methods of combining and administering an HIV antigen, an immunomer, and optionally an adjuvant, so as to enhance the immune response in a mammal. As discussed below in more detail, an immunogenic composition of the invention can be administered locally or systemically by methods well known in the art, including, but not limited to, intramuscular, intradermal, intravenous, subcutaneous, intraperitoneal, intranasal, oral or other mucosal routes.

As disclosed herein, REMUNE has been found to be immunogenic in the majority of patients, although with varying degrees of potency and duration (Example VIII). Therefore, an immunogenic composition comprising HIV devoid of outer envelope proteins combined with IFA adjuvant, such as REMUNE, can be used to induce an immune response in the majority of patients infected with HIV. The immunogenic compositions of the invention enhance the strength and potency of the immune response to an HIV immunogen such as REMUNET, thereby enhancing therapeutic and/or preventative efficacy of a vaccine. An enhanced immune response can be, for example, increased production of HIV-1 specific CD4+ helper T cells, chemokines and/or cytokines, an increase in memory cells, an increase in overall antibody production and more specifically in the ratio of IgG2b production, an increase in cytotoxic T lymphocyte activity, an increase in β-chemokine or IL15 production, and the like. Thus, the immunogenic compositions of the invention can be used to enhance TH1 cytokine profile (high IFNγ, high IgG2/IgG1 ratios). As disclosed herein, the components of the immunogenic compositions of the invention can act in synergy. For example, the immunogenic compositions of the invention can enhance β-chemokine production by eliciting production of a higher concentration of β-chemokine than would be expected by adding the effects of pairwise combinations of components of the immunogenic Memory cells are needed for maintaining long term immunity following the initial acute state of infection. During the contraction phase following an initial acute stage of infection, a significant amount of the immune cells induced against the infectious agent are destroyed by apoptosis, with only the surviving cells remaining able to become

memory cells. Therefore, protecting HIV-specific CD4 and CD8 T cells from apoptosis promotes an increase in both HIV-specific CD4 helper and CD8 CTL memory cells. The immunogenic compositions of the invention can be used to increase memory cells, thereby promoting long term helper functions and cell-mediated immunity. The immunogenic compositions of the invention can be used to increase the number of memory cells by decreasing apotosis or by stimulating factors that promote survival of memory cells.

The immunogenic compositions of the invention can be used to shift a TH2 to a THI response, thereby increasing cell-mediated immune responses, including a stronger CD8+ response. Thus, the immunogenic compositions of the invention can be used to strengthen the immune response in a patient, who otherwise is only responding weakly, and convert the response to cell-mediated immunity. The immunogenic compositions of the invention can thus be used to increase the strength and duration of an immune response in a patient that would have responded weakly to a similar HIV antigen as that used in the immunogenic composition.

An immunogenic composition of the invention is effective in enhancing an immune response, for example, enhanced β-chemokine and/or IL15, IFN, IL2, TNFα production, increased HIV-specific CD4 helper cells, IgG2b antibody production, HIV-specific cytotoxic T lymphocyte (CTL) production, IFNγ production by CD4+ cells and CD8 T cells, and the like, in a mammal administered the composition. As described in U.S. application Ser. No. 09/565,906, filed May 5, 2000, and WO 00/67787, each of which is incorporated herein by reference, and in Examples I and III, below, production of the β-chemokine RANTES can be detected and quantitated using an ELISA assay of supernatants of T cells (such as lymph node cells or peripheral blood cells) from mammals administered the composition. In order to determine antigen-specific β-chemokine production, T cells from an immunized mammal can be stimulated with HIV antigen in combination with antigen-presenting thymocytes, and the β-chemokine levels measured in the supernatant. In order to determine non-specific β-chemokine production, either T cell supernatant or a blood or plasma sample from an immunized mammal can be assayed. Similarly, production of other β-chemokines, such as MIP-1α and MIP-1β, can be detected and quantitated using commercially available ELISA assays, according to the manufacturer's instructions.

Methods of measuring cytokine production, including inteferon, IL15, IL2, TNFα, IL10 and IL7, by ELISPOT, ELISA, or intracellular cytokine staining are well known to those skilled in the art (see, for example, Robbins et al., AIDS 17:1121-1126 (2003)).

An immunogenic composition of the invention can further be capable of enhancing HIV-specific IgG2b antibody production in a mammal administered the composition. High levels of IgG2b antibodies, which are associated with a Th1 type response, are correlated with protection against HIV infection and progression to AIDS. Thus, the invention provides compositions that can increase a TH1 response.

An immunogenic composition of the invention can further be capable of enhancing HIV-specific cytotoxic T lymphocyte (CTL) responses in a mammal administered the composition. An immunogenic composition of the invention can increase IFN-γ production by both CD4+ T cells and CD8+ T cells.

IFN-γ production by CD4+ T cells is characterized as a classic CD4 helper response important to cell-mediated immunity. CD4+ T cells producing both IFN and IL2 may be most effective. IFN-γ production by CD8+ T cells is representative of a cytotoxic T lymphocyte (CTL) response, and is highly correlated with cytolytic activity. Cells producing both IFN and TNFα may be most effective. CTL activity is an important component of an effective prophylactic or therapeutic anti-HIV immune response. Methods of determining whether a CTL response is enhanced following administration of an immunogenic composition of the invention are well known in the art, and include cytolytic assays and LPA assays (described, for example, in Deml et al. supra (1999); see Example III), and ELISA and ELISPOT assays for CD8-specific IFN-γ production (see U.S. application Ser. No. 09/565,906 and WO 00/67787 and Examples I and II below), intracellular staining and FACS analysis using a myriad of antibodies against cell surface markers.

The invention also provides a method of immunizing an individual. The method consists of enhancing the immune response in an individual by administering to a mammal an immunogenic composition containing an HIV antigen, an immunomer, and optionally an adjuvant. The components of the immunogenic composition can be administered in any order or combination, such that the immunogenic composition is formed ex vivo or in vivo.

In a particular embodiment, the HIV antigen, immunomer and optional adjuvant are administered simultaneously or at about the same time, in about the same site. However, administering the components within several minutes or several hours of each other can also be effective in providing an immunogenic composition that an immune response. Additionally, administering the components at different sites in the mammal can also be effective in providing an immunogenic composition that enhances an immune response. One skilled in the art can readily determine a suitable time and location to separately administer components, that is, the HIV antigen, immunomer and optional adjuvant components, to provide a sufficient immune response by administering the separate components at various times and locations and measuring the immune response. The immunogenic composition can also be administered multiple times, if desired, for example, 2 or more, 3 or more, 4 or more, 5 or more, 6 or more, 7 or more, 8 or more, 9 or more, or 10 or more, or any desired number of times to stimulate or enhance an HIV-specific immune response.

The immunogenic compositions of the invention can be administered to a human to inhibit AIDS, such as by preventing initial infection of an individual exposed to HIV, reducing viral burden in an individual infected with HIV, prolonging the asymptomatic phase of HIV infection, increasing overall health or quality of life in an individual with AIDS, or prolonging life expectancy of an individual with AIDS. As disclosed herein, administration to a mammal of an immunogenic composition containing an HIV antigen, an isolated nucleic acid molecule containing an immunomer, and optionally an adjuvant stimulates immune responses correlated with protection against HIV infection and progression to AIDS.

In particular, the immunogenic compositions enhance the immune response more effectively than would be expected by combination of any of the individual components or, in a three component composition containing HIV antigen, immunomer and adjuvant, any two components of the immunogenic compositions. Additionally, the immunogenic compositions promote strong Th1 type immune responses, including both Th1 type cytokines (for example, IFN-γ) and Th1 type antibody isotypes (for example, IgG2b). Thus, the immunogenic compositions of the invention will be effective as vaccines to prevent HIV infection when administered to seronegative individuals, and to reduce viral burden, prolong the asymptomatic phase of infection, and positively affect the health or lifespan of a seropositive individual.

Individuals who have been exposed to the HIV virus usually express in their serum certain antibodies specific for HIV. Such individuals are termed “seropositive” for HIV, in contrast to individuals who are “seronegative.” The presence of HIV specific antibodies can be determined by commercially available assay systems.

At the present time, serological tests to detect the presence of antibodies to the virus are the most widely used method of determining infection. Such methods can, however, result in both false negatives, as where an individual has contracted the virus but not yet mounted an immune response, and in false positives, as where a fetus may acquire the antibodies, but not the virus from the mother. Where serological tests provide an indication of infection, it may be necessary to consider all those who test seropositive as in fact, being infected. Further, certain of those individuals who are found to be seronegative may in fact be treated as being infected if certain other indications of infection, such as contact with a known carrier, are satisfied.

The immunogenic compositions of the invention can be administered to an individual who is HIV seronegative or seropositive. In a seropositive individual, it may be desirable to administer the composition as part of a treatment regimen that includes treatment with anti-viral agents, such as protease inhibitors. Anti-viral agents and their uses in treatment regimens are well known in the art, and an appropriate regimen for a particular individual can be determined by a skilled clinician.

As described in U.S. application Ser. No. 09/565,906 and WO 00/67787 and disclosed herein and in Example IV, below, administration of the immunogenic compositions of the invention to a primate fetus or to a primate neonate results in the generation of a strong anti-HIV immune response, indicating that the immune systems of fetuses and infants are capable of mounting an immune response to such compositions which should protect the child from HIV infection or progression to AIDS. Accordingly, the immunogenic compositions of the invention can be administered to an HIV-infected pregnant mother to prevent HIV transmission to the fetus, or to a fetus, an infant, a child or an adult as either a prophylactic or therapeutic vaccine.

The dose of the immunogenic composition, or components thereof, to be administered in the methods of the invention is selected so as to be effective in stimulating the desired immune responses. Generally, an immunogenic composition formulated for a single administration contains between about 1 to 200 μg of protein antigen. An immunogenic composition generally contains about 100 μg of protein antigen for administration to a primate, such as a human. As described in U.S. application Ser. No. 09/565,906 and WO 00/67787 and disclosed herein and shown in Example IV, below, about 100 μg of HIV antigen in an immunogenic composition elicits a strong immune response in a primate. About 10 μg of HIV antigen is suitable for administration to a rodent. One skilled in the art can readily determine a suitable amount of HIV antigen to include in an immunogenic composition of the invention sufficient to stimulate an immune response.

The immunogenic compositions of the invention can further contain from about 5 μg to about 100 μg of an immunomer, and can contain up to 10 mg of immunomer, if desired. For example, in a dose for administration to a human, the dose can be about 0.01 mg to about 5 mg, for example, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.7 mg, or about 2 mg. The amount of immunomer to be administered is generally about 0.1 mg/kg to about 0.25 mg/kg up to about 5 mg/kg, and can be, for example, about 0.2, about 0.3, about 0.4, about 0.5, about 0.6, about 0.7, about 0.8, about 0.9, about 1, about 1.2, about 1.5, about 1.7, about 2, about 2.5, about 3, about 3.5, about 4, about 4.5, or about 5 mg/kg. The amount of immunomer can also be about 0.2 μg/kg, about 0.5 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 22 μg/kg, about 25 μg/kg and the like. As described previously in U.S. application Ser. No. 09/565,906 and WO 00/67787, a ratio of at least 5:1 by weight of nucleic acid molecule to HIV antigen was more effective than lower ratios for eliciting immune responses. One skilled in the art can readily determine an appropriate or optimized ratio of immunomer to HIV antigen for eliciting an immune response. For example, the ratio can be varied and the immune response measured by methods disclosed herein to determine a suitable or optimized ratio of immunomer to HIV antigen. In rodents, an effective amount of an immunomer in an immunogenic composition is from 5 μg to greater than 50 μg, such as about 100 μg. In primates, about 500 μg of an immunomer is suitable in an immunogenic composition. Those skilled in the art can readily determine an appropriate amount of immunomer to elicit a desired immune response.

As with all immunogenic compositions, the immunologically effective amounts are determined empirically, but can be based, for example, on immunologically effective amounts in animal models, such as rodents and non-human primates. Factors to be considered include the antigenicity, the formulation (for example, volume, type of adjuvant), the route of administration, the number of immunizing doses to be administered, the physical condition, weight and age of the individual, and the like. Such factors are well known in the vaccine art and it is well within the skill of immunologists to make such determinations without undue experimentation.

The immunogenic compositions of the invention can be administered locally or systemically by any method known in the art, including, but not limited to, intramuscular, intradermal, intravenous, subcutaneous, intraperitoneal, intranasal, oral or other mucosal routes. The immunogenic compositions can be administered in a suitable, nontoxic pharmaceutical carrier, or can be formulated in microcapsules or as a sustained release implant. The immunogenic compositions of the invention can be administered multiple times, if desired, in order to sustain the desired immune response. The appropriate route, formulation and immunization schedule can be determined by those skilled in the art.

It is understood that modifications which do not substantially affect the activity of the various embodiments of this invention are also included within the definition of the invention provided herein. Accordingly, the following examples are intended to illustrate but not limit the present invention.

### EXAMPLE I

### Elicitation of Cytokine, Antibody and Chemokine Responses by HIV Immunogenic Compositions

This example is designed to show that immunogenic compositions containing an HIV antigen, immunomer and an adjuvant, are potent stimulators of IFN-γ production (a Th1 (CD8) and Th2 (CD4 helper) cytokine), antibody responses and β-chemokine production in a mammal. Therefore, immunogenic compositions containing an HIV antigen, an immunomer and an adjuvant mediate potent immune responses of the types that are important in protecting against HIV infection and disease progression, indicating that these compositions will be effective prophylactic and therapeutic vaccines. Immunomers. Immunomers are synthesized as described previously (Kandimalla et al., Bioorg. Med. Chem. 9:807-813 (2001); Yu et al., Nucl. Acids Res. 30:4460-4469 (2002); Yu et al., Bioorg. Med. Chem. 11:459-464 (2003); Bhagat et al., Biochem. Biophys. Res. Comm. 300:853-861 (2003), and Yu et al. Biochem. Biophys. Res. Comm. 297:83-90 (2002); Yu et al., Nucl. Acids Res. 30:1613-1619 (2002); Yu et al., J. Med. Chem. 45:4540-4548 (2002); Kandimalla et al., Bioconjugate Chem. 13:966-974 (2002); Yu et al., Bioorganic Med. Chem. Lett. 10:2585-2588 (2000); Agrawal and Kandimalla, Trends Mol. Med. 8:114-121 (2002)).

Immunizations. The HIV-1 antigen is prepared essentially as described previously (WO (00/67787). Briefly, the HIV-1 antigen is prepared from virus particles obtained from cultures of a chronically infected Hut 78 with a Zairian virus isolate (HZ321) which has been characterized as subtype “M,” containing an env A/gag G recombinant virus (Choi et al., AIDS Res. Hum. Retroviruses 13:357-361 (1997)). The gp120 is depleted during the two-step purification process. The antigen is inactivated by the addition of β-propiolactone and gamma irradiation at 50 kGγ. Western blot and HPLC analysis is used to show undetectable levels of gp120 in the preparation of this antigen (Prior et al., Pharm. Tech. 19:30-52 (1995)). For in vitro experiments, native p24 is preferentially lysed from purified HIV-1 antigen with 2% triton X-100 and then purified with Pharmacia Sepharose™ Fast Flow S resin. Chromatography is carried out at pH=5.0, and p24 is eluted with linear salt gradient. Purity of the final product is estimated and is generally found to be >99% by both SDS (sodium dodecyl sulfate) electrophoresis and reverse phase high pressure liquid chromatography. The immunomer is added to the diluted HIV-1 antigen in a volume of at least 5% of the final volume.

CFA (complete Freund's adjuvant) is prepared by resuspending mycobacterium tuberculosis H37RA (DIFCO, Detroit, Mich.) at 10 mg/ml in IFA (DIFCO, Detroit, Mich.). IFA or ISA 51® is formulated by adding one part of the surfactant Montanide 80 (high purity mannide monoleate, Seppie, Paris) to nine parts of Drakeol 6 VR light mineral oil (Panreco, Karnes City, Pa.). The gp120-depleted HIV-1 antigen is diluted in PBS to 200 μg/ml and emulsified with equal volumes of CFA or IFA with or without immunomer.

C57B1 mice, maintained in a pathogen-free facility, are injected intradermally with 100 μl of emulsion. Each animal receives 1-10 μg of the inactivated HIV-1 antigen in either CFA, IFA, 10-100 μg immunomer, or IFA plus 10-100 μg immunomer. Two weeks later, the animals are boosted subcutaneously in the base of the tail using the same regimen, except that the animals primed with HIV-1 antigen in CFA are instead boosted with HIV-1 antigen in IFA. Mice are primed and boosted with HIV-1 antigen in the presence of immunomer. Negative controls are administered as saline or IFA in saline. On day 28, the animals are sacrificed for cytokine, chemokine, and antibody analysis.

ELISA for antigen-specific antibody. Whole blood is collected from immunized animals by heart puncture at the end of the study. The SST tubes are centrifuged at 800 rpm for 20 minutes. Sera are aliquoted and stored at −20° C. until assayed. PVC plates (polychlorinated biphenyl plates, Falcon, Oxnard, Calif.) are coated with native p24 diluted in PBS at 1 μg/ml and stored at 4° C. overnight. Plates are blocked by adding 200 μl per well of 4% BSA in PBS for 1 hour. Sera are diluted in 1% BSA in PBS at 1:100 followed by four-fold serial dilution. 100 μl of diluted sera are added in duplicate and incubated at room temperature for 2 hours. Plates are washed with 0.05% Tween 20 in PBS three times and blotted dry. The detecting secondary antibodies (goat or rat anti-mouse IgG biotin, goat or rat anti-mouse IgG1 biotin, or goat or rat anti-mouse IgG2a biotin, for example, Zymed, San Francisco, Calif.) are diluted in 1% BSA in PBS. 100 μl of diluted secondary antibody is added to each well and incubated at room temperature for another hour. After washing excess secondary antibody, strep-avidin-biotin-HRP (Pierce, Rockford, Ill.) are added at 50 μl per well and incubated for 30 minutes. Plates are washed with 0.05% Tween 20 in PBS three times. ABTS substrate (KPL, Gaithersburg, Md.) is added until a bluish-green color developed. The reaction is stopped by the addition of 1% SDS and the plate is read at absorbance 405 nm.

The antibody response reported as 50% antibody titer is the reciprocal of the dilution equal to 50% of the maximum binding (highest optical reading) for every given sample. The absorbance value (OD @ 405 nm) is plotted against antibody dilution in a log scale, yielding a sigmoidal dose response curve. 50% of the maximum binding is calculated by multiplying the highest OD by 0.5. The 50% value is located on the curve and the corresponding x-axis value is reported as the antibody dilution.

ELISA Assay for Cytokine and Chemokine Analysis. The draining lymph nodes (superficial inguinal and popliteal) are isolated from immunized animals two weeks after the boost. Single cell suspensions from these lymph nodes are prepared by mechanical dissociation using sterile 70 μm mesh screen. T cells are purified from lymph node cells by the panning method. Briefly, petri dishes (100×15 mm) are pre-coated with 20 μg/ml of rabbit anti-mouse IgG for 45 minutes at room temperature. The petri dishes are washed twice with ice cold PBS and once with ice cold 2% human AB serum in PBS. 1×10⁷ lymph node cells are added to pre-washed plates and incubated at 4° C. for 90 minutes. The non-adherent cells (enriched T cells) are then collected and transferred into sterile 50-ml conical tubes. The plates are washed twice and combined with the non-adherent cells. The cells are then centrifuged and cell pellets resuspended in complete media at 4×10⁶ cells/ml (5% human AB serum in RPMI 1640, with 25 mM hepes, 2 mM L-glutamine, 100 μg streptomycin and 5×10⁻⁶ M β-mercaptoethanol).

Gamma-irradiated thymocytes from a C57BL mouse are used as antigen presenting cells. 2×10⁵ enriched T cells and 5×10⁵ thymocytes are added to each well of a 96-round bottom plate. The HIV-1 antigen and native p24 are diluted in complete media at 10 μg/ml while con A is diluted to 5 μg/ml. 100 μl of each antigen or T cell mitogen are added in triplicates. The plates are incubated at 5% CO₂, 37° C. for 72 hours. Supernatants are harvested and stored at −70° C. until assayed. The samples are assayed for IL-4, IFN-γ and RANTES using commercially available kits (for example, Biosource, Camarillo, Calif.) specific for mouse cytokines and chemokines.

Statistical methods. The Mann-Whitney U nonparametric statistic is utilized to compare groups. All p values are two tailed.

Complete Freund's Adjuvant (CFA) is currently the most potent adjuvant known for stimulating cell-mediated immune responses. However, CFA is not an appropriate adjuvant for use in humans because of safety issues. Thus, the combination of immunomer and IFA for use in an HIV immunogenic composition provides for safe and effective vaccines for human therapy.

To examine the dose-related immune response to IFN-γ, C57BL mice are immunized with the inactivated gp120-depleted HIV-1 antigen emulsified in IFA containing different concentrations of immunomer.

To examine whether can also boost the antibody response to an HIV-1 antigen, sera are assayed for total IgG and Th2 isotype (IgG1 and IgG2a) antibody responses to p24 antigen.

Thus, the immunogenic compositions of the invention can be used to enhance β-chemokine production in an individual. Because of the strong correlation between β-chemokine levels and protection from HIV infection and disease progression, the compositions of the invention will be more effective than other described compositions for inhibiting AIDS.

### EXAMPLE II

### Elicitation of CD4 and CD8 Immune Responses by HIV Immunogenic Compositions

This example is designed to show the induction of potent CD4 helper functions, CD8 HIV-specific Th1 type immune responses, and a shift to higher IgG2a/IgG1 antibody ratios following immunization with an immunogenic composition containing an HIV antigen, an immunomer and an adjuvant. Antigen-specific responses by CD8+, cytotoxic T lymphocytes are an important factor in preventing initial HIV infection and disease progression. Thus, this example provides further evidence that the immunogenic compositions of the invention are effective prophylactic and therapeutic vaccines.

HIV antigen, immunomer and IFA are prepared essentially as described in Example I. C57BL mice are immunized essentially as described in Example I, and sacrificed at day 28 for ELISPOT and p24 antibody analysis. p24 antibody analysis is performed essentially as described in Example I.

ELISPOT for gamma-interferon from bulk and purified T cell populations. Single cell suspensions are prepared from spleens of the immunized mice by mincing and pressing through a sterile fine mesh nylon screen in RPMI 1640 (Hyclone, Logan, Utah). The splenocytes are purified by ficoll gradient centrifugation. CD4 and CD8 cells were isolated by magnetic bead depletion. 2×10⁷ cells are stained with 5 μg of either rabbit or rat anti-mouse CD4 or rabbit or rat anti-mouse CD8. Cells are incubated on ice for 30 minutes and washed with ice cold 2% Human AB serum in PBS. Pre-washed Dynabeads (DYNAL, Oslo, Norway) coated with goat anti-mouse IgG are added to the cell suspension and incubated at 4° C. for 20 minutes with constant mixing.

Purified CD4, CD8 and non-depleted splenocytes are resuspended in complete media (5% inactivated Human AB serum in RPMI 1640, Pen-strep, L-glutamine and β-ME) at 5×10⁶ cells/ml and used for ELISPOT assay to enumerate the individual IFN-γ secreting cells. Briefly, 96 well nitrocellulose bottom microtiter plates (Millipore Co., Bedford, U.K.) are coated with 400 ngs per well of rabbit anti-mouse IFN-γ (Biosource, Camarillo, California). After overnight incubation at 4° C., plates are washed with sterile PBS and blocked with 5% human AB serum in RPMI 1640 containing pen-strep, L-glutamine and β-ME) for 1 hour at room temperature. Plates are washed with sterile PBS and 5×10⁵ per well of splenocytes (purified CD4, purified CD8 or non-depleted) were added in triplicate and incubated overnight at 37° C. and 5% CO₂. Cells are cultured with media, OVA (Chicken Egg Ovalbumin, Sigma-Aldrich, St. Louis, Mo.), native p24 or gp120-depleted HIV-1 antigen. CD4 purified and CD8 purified splenocytes are assayed in complete media containing 20 units/ml of recombinant rat IL-2 (Pharmingen, San Diego, Calif.).

After washing unbound cells, 400 ng per well of the polyclonal rabbit anti-mouse IFN-γ are added and incubated at room temperature for 2 hours, then washed and stained with goat anti-rabbit IgG biotin (Zymed, San Francisco, Calif.). After extensive washes with sterile PBS, avidin alkaline phosphatase complex (Sigma-Aldrich, St. Louis, Mo.) is added and incubated for another hour at room temperature. The spots are developed by adding chromogenic alkaline phosphate substrate (Sigma, St. Louis, Mo.), and the IFN-γ cells are counted using a dissection microscope (×40) with a highlight 3000 light source (Olympus, Lake Success, N.Y.).

Statistical Methods. The Mann-Whitney U nonparametric statistic is utilized to compare groups. The Spearman rank correlation is performed to examine relationships between CD4 and CD8 gamma interferon production. All p values are two tailed.

The production of IFN-γ by non-depleted splenocytes, and by purified CD4+ or purified CD8+ populations, is examined. IFN-γ production by CD4+ cells is a characteristic Th1 immune response, whereas IFN-γ production by CD8+ cells is a correlate of cytotoxic T lymphocyte (CTL) cytolytic activity. Total IgG, IgG1 and IgG2b specific for p24 is also examined.

In summary, this Example shows that an immunogenic composition containing an HIV antigen, an immunomer and an adjuvant can be used to generate potent HIV-specific CD4 and CD8 HIV-specific immune responses. The induction of CD4 T helper cells may be pivotal for generation of CD8 effector cells. CD8 T cells can serve as effectors against HIV virus by several mechanisms, including direct cytolytic (CTL) activity, as well as through the release of antiviral suppressive factors, such as β-chemokines and other less well-characterized factors. Accordingly, the compositions described herein are superior to other described compositions for use as HIV vaccines.

### EXAMPLE III

### Comparison of Immune Responses Elicited by Different Immunogenic Compositions and Immunization Schedules

This example is designed to show that a nucleic acid containing an immunomer is more effective in eliciting protective immune responses, including RANTES production and HIV-specific IgG2b antibody production, when administered simultaneously with an HIV antigen and an adjuvant than when used to prime the mammal one week prior to administration of the antigen and adjuvant. This example also shows that a composition containing an HIV antigen, an immunomer and an adjuvant promotes antigen-dependent lymphocyte proliferation more effectively than a composition containing only HIV and IFA.

HIV antigen, immunomers and IFA are prepared essentially as described in Example I. C57bBL mice (at least three per group) are immunized at day 7 and, where indicated, primed at day 0, with the following compositions shown in Table 1.

TABLE 1

| Group | Day 0 | Day 7 |
|---|---|---|
| A | Immunomer | HIV-1 |
| B | | HIV-1 |
| C | Immunomer | HIV-1/IFA |
| D | | HIV-1/IFA |
| E | | HIV-1/IFA/Immunomer |

Animals are sacrificed at day 21 for cytokine, chemokine and antibody analysis, essentially as described in Example I, as well as for analysis of lymphocyte proliferation.

Lymphocyte proliferation assay. Single cell suspensions are prepared from the draining lymph nodes of immunized animals. B cells are depleted from the lymph node cells by panning. Briefly, lymph node cells are incubated with anti-mouse IgG pre-coated petri dishes for 90 minutes. The non-adherent cells (enriched T cells) are collected and resuspended in complete tissue culture media at 4×10⁶ cells/ml. The enriched T cells are cultured with p24 or HIV-1 antigen in the presence of y-irradiated thymocytes at 37° C., 5% CO₂ for 40-48 hours. Samples are pulsed with tritiated thymidine and incubated for another 16 hours. Cells are harvested, and tritiated thymidine incorporation is counted using a β-scintillation counter.

Cytokine production in T cells, for example, IFN-γ and β-chemokines such as RANTES, MIP-1β and MIP-1α is determined using methods well known to those skilled in the art. Serum levels of total IgG, IgG1 and IgG2b specific for p24 are also examined. In addition, T cell proliferative responses to p24 antigen and pg120-depleted HIV are examined.

Thus, the immunogenic compositions of the invention can effectively elicit HIV-specific Th1 cytokine (IFN-γ) and humoral responses (IgG2 antibodies), and can enhance both non-specific and HIV-specific β-chemokine production. These responses to the immunogenic compositions correlate with strong HIV-specific T lymphocyte proliferative responses.

### EXAMPLE IV

### Immunization of a Primate With an HIV Immunogenic Composition

This example is designed to show that immunogenic compositions containing an HIV antigen, an immunomer and an adjuvant are effective in enhancing HIV-specific immune responses in primates.

Three baboon fetuses are injected in utero with an immunogenic composition containing gp120-depleted HIV-1 (100 μg total protein, equivalent to 10 p24 units) in IFA with 500 μg of immunomer. Four weeks later, the fetuses are boosted using the same regimen.

Peripheral blood mononuclear cells from the neonatal baboons are collected, and proliferative responses to p24 and HIV-1 antigen are assayed.

Production of HIV-specific antibodies, cytokines and β-chemokines are also measured in the same baboons. These results show that the types of immune responses elicited by the immunogenic compositions described in Examples I-III, above, for rodents, are also elicited in primates.

These results demonstrate that the HIV immunogenic compositions and methods of the invention are effective in primates in stimulating HIV-specific immune responses. Furthermore, these results demonstrate that fetuses and infants are able to elicit strong HIV immune responses to the immunogenic compositions of the invention, indicating that these compositions will be useful for preventing maternal transmission of HIV and as pediatric vaccines.

### EXAMPLE V

### Immune Response to Vaccination With Inactivated gp120 Depleted HIV Immunogen Combined With Immunomer in a Mouse Model

This example describes characterization of the ability of an immunomer to enhance the immunogenecity of HIV-1 antigen and HIV-1 Immunogen (antigen emulsified in IFA) in a mouse model.

C57BL/6 mice (6-8 weeks of age) are injected as indicated below. The number per group is generally at least 8-10 mice.

1) PBS

2) Immunomer at 30 μg per mouse=1.5mg/kg

3) Immunomer (highest dose of 90 μg)=4.5 mg/kg

4) HIV-1 immunogen (10 μg)

5) HIV-1 immunogen+Immunomer (10 μg, 30 μg, 90 μg)

6) HIV-1 immunogen+Immunomer 30 μg

The gp120 depleted HIV-1 antigen is diluted in phosphate buffered saline (PBS) to concentration of 200 μg/ml and emulsified in equal volumes of IFA, with and without of immunomer. The immunomer is added to the diulted HIV-1 antigen prior to emulsion in a volume of at least 5% of the final volume.

An initial single intradermal injection is performed at time 0 followed by intradermal injection after 2 weeks. The mice are sacrificed 2 weeks after the booster injection. The HIV-1 immunogen used is inactivated gp120 depleted HIV-1 antigen in IFA.

Immunological analyses. Fresh splenic mononuclear cells are isolated and stimulated in vitro for 4 days (Davis et al., J. Immunol. 160:870-876 (1998). The isolated cells are stimulated in medium alone; with native p24 antigen; or with HIV-1 antigen.

The production of various cytokines are evaluated using ELISA methods. Exemplary cytokines to be assayed include, for example, IFNγ, IL-12, IL-4, IL-5, IL-10, MIP1α, MIP1β, RANTES, α-defensin as disclosed herein and described previously, and are assayed by methods well known to those skilled in the art.

P24 antigen- and HIV-1 antigen-specific IFNγ production in CD4 and CD8 lymphocytes is evaluated in ELISPOT assays, as described in Examples I and II.

P24 antigen-, HIV-1 antigen, and LPS-specific lymphocyte proliferation are evaluated in a standard proliferation assay using well known methods.

### EXAMPLE VI

### In Vitro Effect of Immunomer on HIV Specific Immune Response Generated by PBMCs From HIV-Infected Patients Previously Immunized With HIV-1 Immunogen

This example describes evaluation of the ability of an immunomer to increase HIV-specific immune responses in vitro in peripheral blood mononuclear cells (PBMC) of patients who have been treated with inactivated gp120 depleted HNW-1 antigen in IFA (REMUNE).

The following groups of patients are examined: 15 HIV-infected, HAART+REMUNE-treated patients; 15 HIV-infected, HAART-treated patients. The patients are 10 matched for disease duration, CD4 counts, HIV viremia, and absence/presence of protease inhibitor (PI). Whole blood (530 ml) is drawn by venipuncture in EDTA-containing tubes for subsequent analysis. Immunomer is added to the PBMCs at the following concentrations: 0.1 μg/ml, 1.0 μg/ml, 10.0 μg/ml.

Responses specific to various antigens are measured, for example, HIV antigens 15 p24 antigen, HIV-1 antigen, env peptides, gag peptides; and flu (control antigen). Other HIV antigens can also be measured, if desired. Antigen-specific IFNγ-production in CD4 and CD8 lymphocytes is evaluated in ELISPOT assays, as described in Examples I and II. Antigen-specific lymphocyte proliferation is also evaluated in a standard proliferation assay.

The production of RANTES, a defensin is evaluated by intracellular staining in CD8+ with fluroescence activated cell sorting (FACS) methods. If desired, other cytokines or other cell types can be assayed.

### EXAMPLE VII

### In Vivo Effect of Immunomer on HIV Specific Immune Response in a Trimera Murine Model

This example describes the use of a Trimera mouse model for determining the effect of an HIV immunogenic composition containing immunomers.

A Trimera mouse model is used to test the effect of immunomers when combined with an HIV antigen. Both induced immune responses as well as protective immunity can be monitored. Trimera mice are generated as described previously (Reisner and Dagan, Trends Biotechnol. 16:242-246 (1998); Ilan et al., Curr. Opin. Mol. Ther. 4:102-109 (2002); U.S. Pat. No. 6,254,867; WO 97/47654). Briefly, a normal mouse host is rendered immuno-incompetent by a lethal split-dose total body irradiation. The mice are then radioprotected by T-cell-depleted murine SCID bone marrow and converted to Trimera mice by intraperitoneal injection of human peripheral blood mononuclear leukocytes (PBMCs). Engraftment of the human cells in the Trimera mice is verified by fluorescence activated cell sorting (FACS) analysis of human T cell markers such as CD3 or others.

Trimera mice are infected with HIV as a model of AIDS. Briefly, Trimera mice are infected with one or more strains of HIV-1. Control animals are Trimera mice injected with medium only (without HIV-1) and mice not injected with PBMCs. Mice are evaluated at various time points for HIV-1 infection by determining the levels of plasma HIV-1 RNA, the presence of proviral DNA, and active virus in coculture experiments. The presence of proviral HIV-1 DNA is demonstrated by PCR of an HIV-1 sequence such as gag.

To test an immunogenic composition containing an immunomer for stimulation of an immune response, Trimera mice are injected with gp120-depleted HIV-1, with or without at least one immunomer and with or without adjuvant. Various ratios of antigen and immunomer can be used, for example, as described in Example V, and tested for an optimized immune response. Alternatively, the compositions above are pulsed into human autologous monocyte-derived dendritic cells (DCs), and these DCs are injected into the Trimera mice. Optionally, the mice can be boosted with a similar composition.

Following immunization, blood and peritoneal lymphocytes are collected. The presence of immunoglobulins specific for HIV antigens is determined. In addition, specific cellular anti-HIV responses are determined in human lymphocytes isolated from the mice. For example, IFNγ production in human lymphocytes recovered from Trimera mice is determined following exposure to HIV-1 antigens. The enhanced immunogenic response to HIV antigen in the presence of immunomer is determined.

Protective immunity is monitored in a similar fashion, except that mice are immunized with the various compositions prior to inoculation with infective HIV. The ability of the various compositions to influence the level of ensuing viremia is measured, as described above. The most efficacious vaccine is the one providing the most effective control of circulating virus and/or prolonging survival.

### EXAMPLE VIII

### Immunization of HIV Infected Patients With REMUNE™

This example describes immunization of HIV infected patients with REMUNE™ (GP120 depleted HIV-1 antigen in IFA) and demonstrates that the majority of patients can mount immune responses, although at variable strengths and durations. The objective of this particular study was to evaluate HIV-1 specific immunologic responses following treatment with REMUNE in combination with highly active retroviral therapy (HAART) (indinavir/ZDV/3TC) compared to Incomplete Freunds Adjuvant (IFA) plus HAART.

The study protocol was a randomized, double blind, two arm, parallel group, adjuvant controlled, multicentre study. The number of subjects (total and for each treatment) was 52 patients randomized, with 43 evaluable patients in intent to treat analysis (22 REMUNE+HAART; 21 IFA+HAART). The diagnosis and criteria for inclusion was HIV-1 infected patients with CD4 counts >350 cells/μL with no previous use of HIV protease inhibitors or lamivudine (3TC). The test product, dose, and mode of administration were REMUNE (HIV-1 Immunogen); 10 units (equal to 10 μg/ml p24 content), volume of 1.0 ml given IM (batch No. 8155-015 and 8155-017). For duration of treatment, patients received HAART for 32 weeks. REMUNE or IFA placebo (control) was given at weeks 4, 16 and 28. The reference therapy, dose, and mode of administration were adjuvant controlled; IFA placebo was used (batch nos: 8144-006 and 8160-005).

The primary efficacy criteria was lymphocyte proliferative (L?) responses to HIV-1 antigen stimulation in peripheral blood mononuclear cells (PBMC). Secondary efficacy criteria included LP response to native p24 and BaL HIV-1 antigen stimulation in PBMC; chemokine response to native p24 and HIV antigen stimulation in PBMC; gag CTL activity (in a subset of patients); changes in CD4 cell count and percent CD4; changes in viral load measured as plasma RNA and PBMC DNA; and DTH skin test response to HIV-1 and p24 antigens. The statistical methods used were Fisher's Exact Test (two-tailed) in an intent-to-treat analysis and two-sided Mann-Whitney test.

The primary analysis defined response rate as stimulation index (SI) to HIV-1 antigen five fold over baseline at two time points. Results showed that there were 14/22 (64%) responders in the REMNNE +HAART group and 4/21 (19%) responders in the IFA +HAART group (p=0.005). Secondary analyses defined response rate as SI to BAL type HIV-1 antigen and/or p24 antigen three fold over baseline at two time points. Results showed there were 15/22 (68%) responders in the REMUNE+HAART group and 5/21 (24%) responders in the IFA +HAART group (p=0.006). The magnitude of the LP response to HIV-1 (HZ321) antigen among subjects receiving REMUNE+HAART was greater than among those receiving IFA+HAART (p=0.0028), defined as the ratio for each subject of the geometric mean SI measured after the first injection to the geometric mean of pretreatment values.

There was a statistically significant greater LP response rate to native p24 (p=0.0002) and to HIV-1 BaL antigen (p=0.007) in the REMUNE+HAART compared to IFA+HAART group. There were no differences in LP response to recall antigens (candida, streptokinase, tetanus) between the two groups. MIP-1β production by PBMC stimulated with HIV-1 antigen was significantly augmented in the REMUNE+HAART group (p+0.0007 at week 32) compared to the EPA+HAART group. There was a greater DTH skin test response rate in the REMUNE+HAART group compared to the IFA+HAART group for HIV-1 (53% vs. 9%) and native p24 antigens (47% vs. 0%/o).

The administration of REMUNE plus ZDV/3TC/indinavir resulted in a significant stimulation of lymphocyte proliferation (LP) responses to HIV-1 antigen in terms of both the number of responders and magnitude of the response. For response rate, defined as stimulation index to HIV-1 antigen five-fold over baseline at two time points, there was a significantly higher number of responders (p=0.005) in the REMUNE plus ZDV/3TC/indinavir group (14/22, 64%) than in the IFA plus ZDV/3TC/indinavir group (4/21, 19%).

A high percentage of subjects receiving REMUNE generated strong LP response to native p24 antigen, demonstrating that REMUNE can generate responses specifically to the more conserved core antigens of HIV. Treatment with IFA did not stimulate HIV-1 specific immune responses to any antigen. REMUNE plus ZDV/3TC/indinavir produced a significantly higher lymphocyte proliferation response rate to purified native p24 (p=0.0002).

Administration of REMUNE stimulated LP responses to the HIV-1 (HZ321) immunizing antigen as well as to an HIV-1 antigen that is clade B, HIV-1 BaL antigen, demonstrating that the immune responses generated by REMUNE are cross-clade and not limited to the immunizing agent. REMUNE plus ZDV/3TC/indinavir produced a significantly higher lymphocyte proliferation response rate to HIV-1 BaL antigens (p=0.007) compared to IFA plus ZDV/3TC/indinavir.

The production of antigen stimulated MIP-1β was significantly increased in the REMUNE plus ZDV/3TC/indinavir group throughout the study (p=0.0007 at week 32) and did not change in the IFA plus ZDV/3TC/indinavir group during the study. Subjects in both groups showed significant increases in CD4 cell count and significant decreases in plasma HIV RNA and proviral HIV DNA copy number. There was a trend of less risk of relapse in the REMUNE plus ZDV/3TC/indinavir group in an analysis of time to HIV RNA relapse; 6/22 (27%) of REMUNE plus ZDV/3TC/indinavir subjects relapsed between Week 16 and 32 versus 12/21 (57%) of the IFA plus ZDV/3TC/indinavir subjects (p=0.08 by log-rank test). A stronger, more durable response is expected by administering immunogenic compositions of the invention that include an HIV antigen such as REMUNE and one or more immunomers.

These results demonstrate that REMUNE stimulates an immune response in the majority of patients.

### EXAMPLE IX

### Immunization of HIV Infected Patients With REMUNE™ and Immunomers

This example describes immunization of HIV infected patients with REMUNE and immunomers.

A study is conducted with the objective of evaluating HIV-1 specific immunologic responses following treatment with REMUNE in combination with immunomers and/or highly active retroviral therapy (HAART) (indinavir /ZDV /3TC) compared to Incomplete Freunds Adjuvant (IFA) plus immunomers and/or HAART.

The methodology uses a randomized, double blind, two arm, parallel group, adjuvant controlled study. The diagnosis and criteria for inclusion of HIV-1 infected patients are patients with CD4 counts >350 cells/μL with no previous use of HIV protease inhibitors or lamivudine (3TC). Other criteria for selecting patients can also be used. The test product, dose, and mode of administration are REMUNE (HIV-1 Immunogen); 10 units (equal to 10 μg/ml p24 content), volume of 1.0 ml given IM. A dose of immunomer between about 1 to 5 mg/kg is administered. Other doses of immunomer, either greater or lower, can also be tested for effective enhancement of an immune response. For duration of treatment in patients being treated with HAART, patients receive HAART for 32 weeks. REMUNE or IFA placebo (control) and immunomer is given at weeks 4, 16 and 28. The reference therapy, dose, and mode of administration, are adjuvant control, in which IFA placebo is used.

The criteria for evaluation is similar to that described in Example VIII for efficacy and safety. In addition, assays for determining an immune response can be included, for example, interferon ELISPOT, IgG1/IgG2 antibody ratios, ELISA assays for production of cytokines, lymphocyte proliferation assay, stimulation of spleen cells, and the like, as disclosed herein and described in Examples I-III and V.

The combination of immunomers with REMUNE or other HIV antigen is expected to enhance the immune response in comparison to HIV antigen without immunomers. Thus, the immune response in the present example is expected to be stronger and/or have a longer duration than that observed in Example VIII.

This example describes the enhanced effect of administering HIV antigen with immunomer to stimulate an immune response in HIV infected patients.

### EXAMPLE X

### HIV-1 Antigen With an Immunomer Elicits HIV-Specific Immunity

This example describes the use of HIV antigen and an immunomer to stimulate HIV-specific immunity.

HIV-1 Immunogen is a gp120-depleted whole killed virus vaccine candidate formulated with Incomplete Freund's Adjuvant (IFA), previously reported to induce HIV-1 specific immune responses; synthetic oligonucleotides containing immunostimulatory cytosine-guanine (CpG) dinucleotide motifs are potent stimulators of cell-mediated immune responses. The possibility of generating enhanced immunogenicity of HIV-1 Immunogen in combination with an immunomer adjuvant (Amplivax™) was studied in a mouse model. In subsequent experiments, it was verified that HIV-specific immune responses could be elicited by the gp120-depleted whole killed virus without IFA (HIV-1 Antigen)+Amplivax™.

In these studies, the HIV-1 immunogen used was a gp120-depleted whole killed virus vaccine formulated with Incomplete Freund's Adjuvant (IFA). The experiments were performed essentially as described in Example V. This immunogen induces HIV-specific immune responses. Amplivax™ is an immunomodulatory oligonucleotide, also referred to herein as an immunomer, containing a novel structure and a synthetic immunomodulatory motif. This immunomer induces distinct immunostimulatory profiles.

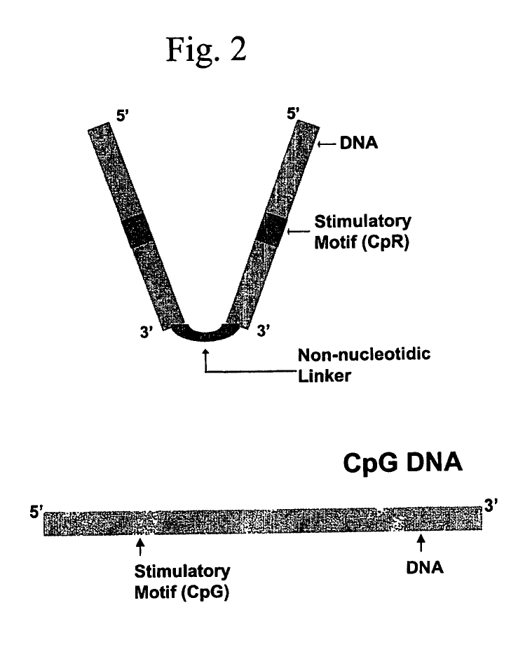

FIG. 2 shows a schematic diagram of the Amplivax™ immunomer, also referred to as HYB2055. HYB2055 is a second-generation immunomodulatory oligonucleotide (IMO) consisting of a novel structure and synthetic, CpR, immunostimulatory motif. This immunomer stimulates the immune system by signaling through TLR9 and induces Th1 immune responses. The immunomer shows enhanced metabolic stability. A phase I trial in healthy volunteers has been completed.

These mouse studies were initiated to evaluate the ability of Amplivax™ (HYB2055 ) to enhance immmunogenicity of HIV-1 whole killed vaccine in IFA (HIV-1 immunogen) in a mouse model. These studies also examined whether HIV-specific immune responses can be elicited by the whole killed virus without IFA (HIV-1 antigen) used in combination with Amplivax™.

Briefly, C57/BL6 mice were immunized subcutaneously (SC) or intramuscularly (IM) (day 0 and 14) with 10 μg of HIV whole killed vaccine in incomplete Freund's adjuvant (IFA) (HIV-1 Immunogen) plus three doses of Amplivax™ (90, 30 or 10 μg/mouse) or with HIV whole killed vaccine (HIV-1 Antigen without IFA, 10 μg/mouse) and Amplivax™ (90 μg/mouse). Animals immunized with HIV-1 Immunogen, with Amplivax™ alone, or with PBS were used as controls (8-10/group). Mouse Immunomer, HYB 2048, was used as a reference compound. Mice were sacrificed on day 28. HIV-1 antigen- and p24-stimulated cytokine production and IFNγ-secreting T cells were evaluated in fresh splenic mononuclear cells. P24 antibody production was evaluated in serum.

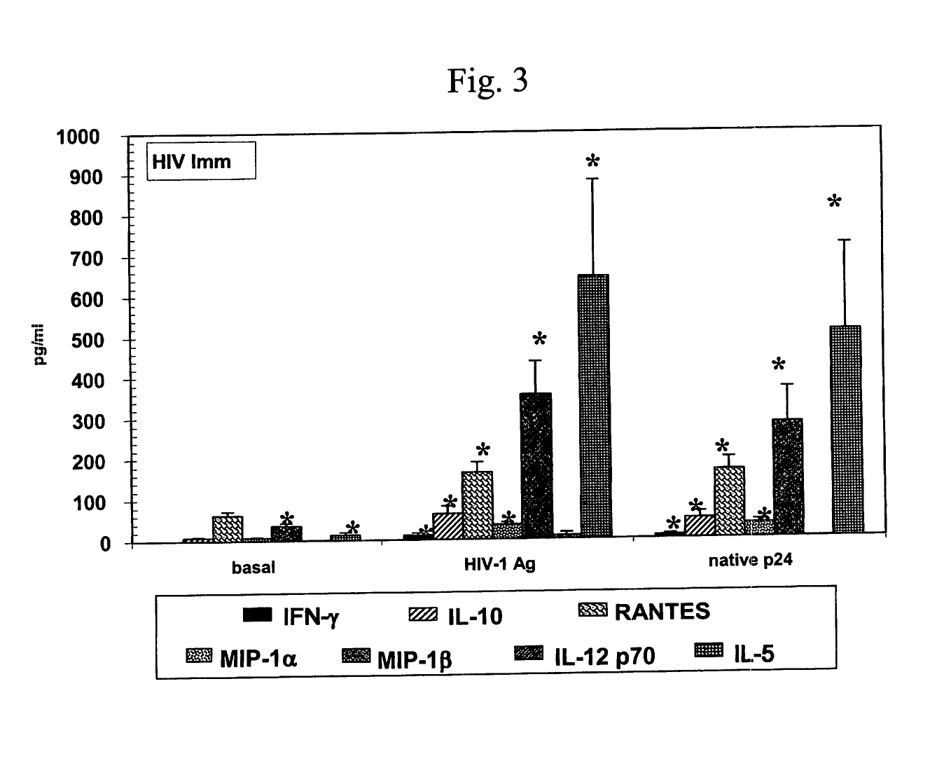

As shown in FIG. 3, HIV-1 Immunogen induces HIV-specific RANTES, MIP1α, MIP1β, IL-10 and IL-5 production. Table 2 shows that the combination of HIV-1 Immunogen and Amplivax™ shifted cytokine profile towards Th1 type responses. Immunogen was administered SC. The values shown are mean values.

TABLE 2

| | IFN-γ | RANTES | MIP-1α | MIP-1β | IL-10 | IL-5 | Cytokine |
|---|---|---|---|---|---|---|---|
| | pg/ml | pg/ml | pg/ml | pg/ml | pg/ml | pg/ml | profile |
| HIV-1 | 12 | 101 | 28 | 317 | 67 | 644 | Th2 |
| Immunogen alone | | | | | | | |
| HIV-1 | 1783 | 700 | 83 | 438 | 257 | 6 | Th1 |
| Immunogen plus | | | | | | | |
| Amplivax | | | | | | | |
| Amplivax alone | 0.06 | 107 | 27 | 91 | 3 | — | — |

A similar analysis is shown in Table 3, which also shows the ratio of IFN-γ to IL-5. Stimulation was with HIV-1 antigen. IFN-γ and IL-5 were measured by ELISA.

TABLE 3

| | IFN-g | IL-5 | IFN-g/IL-5 | Cytokine |
|---|---|---|---|---|
| | pg/ml | pg/ml | pg/ml | Profile |
| HIV-1 Immunogen | 12 | 644 | 0.02 | Th2 Type |
| HIV-1 Immunogen + | 1828 | 5.7 | 321 | Th1 Type |
| Amplivax | | | | |
| Amplivax | .064 | 5.4 | — | — |

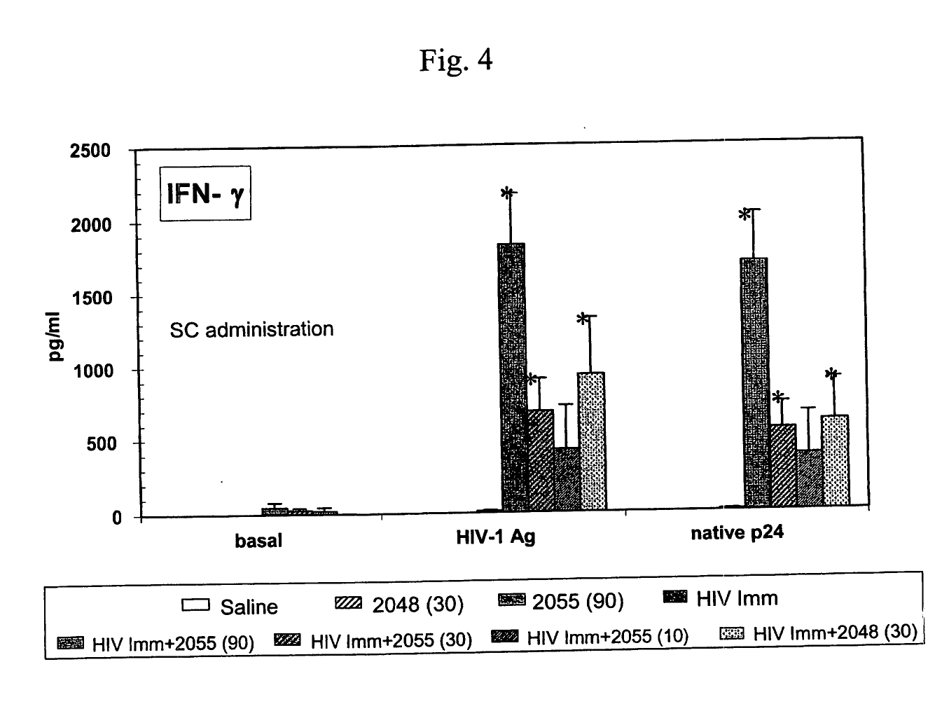

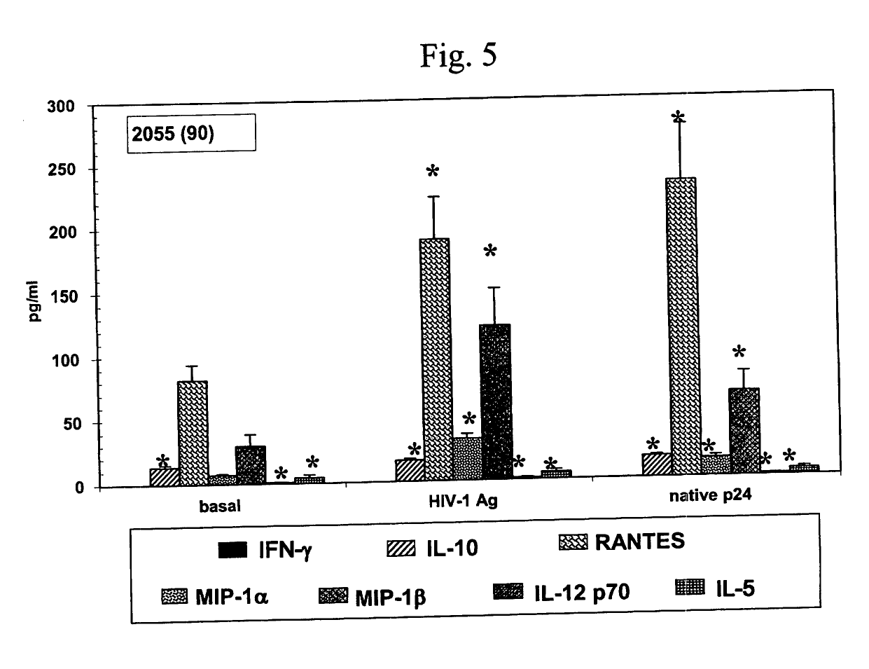

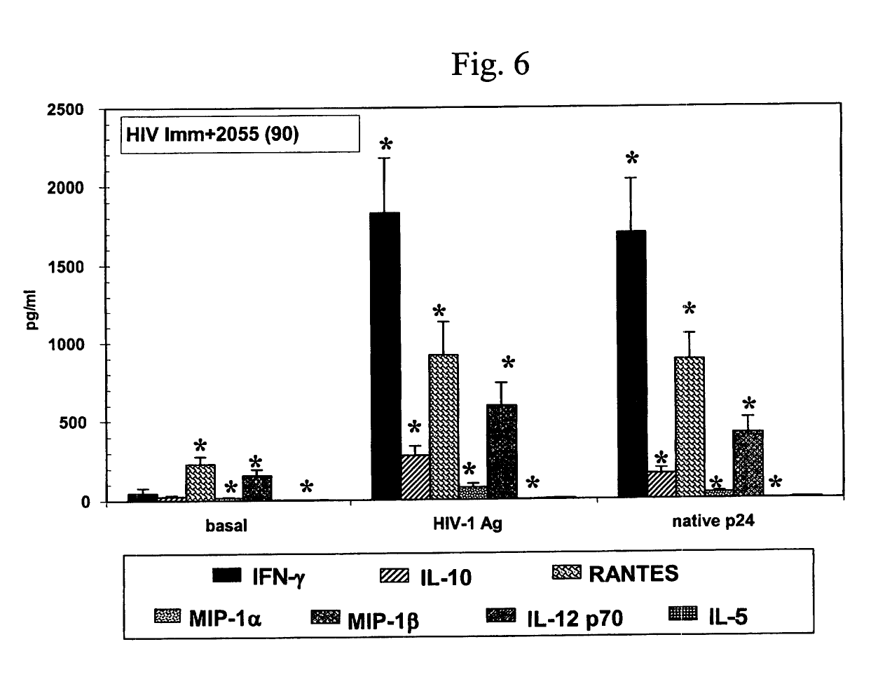

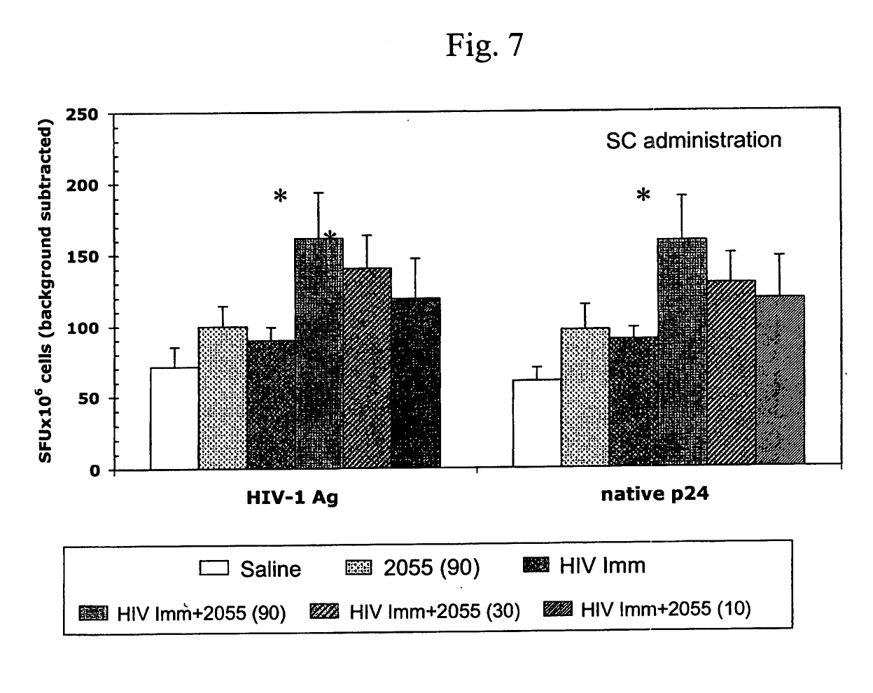

FIG. 4 shows HIV-specific IFNγ production is enhanced by Amplivax™ in a dose dependent manner. The amount of immunomer used is shown in parentheses (μg/mouse). Similar results were seen for RANTES, MIP1α, MIP1β and IL-10. FIG. 5 shows the effect of Amplivax™ on HIV-1 immunogen-induced production levels of RANTES, MIP1α, MIP1β, IL-10 and IL-5. FIG. 6 shows that HWV-specific IFNγ production is enhanced by Amplivax™ (data shown for 90 μg/mouse Amplivax™). Similar results were found for RANTES, MIP1α, MIP1β and IL-10. As shown in FIG. 7, Amplivax™ has an enhancing effect on HIV-specific IFNγ-secreting T cells in an Elispot assay. Immunogen was administered subcutaneously. The amount of immunomer used is shown in parentheses (μg/mouse).

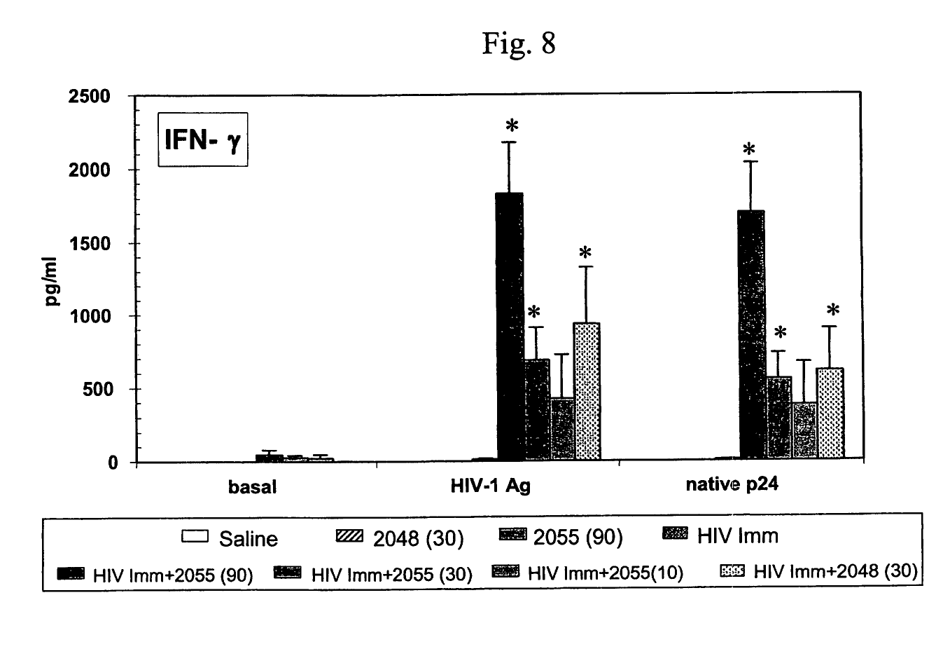

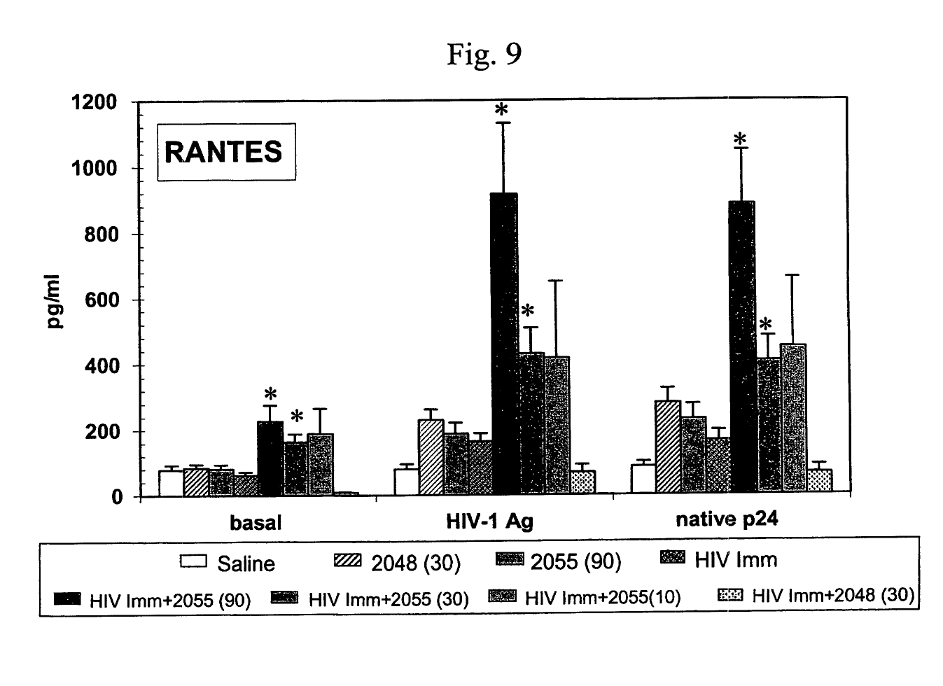

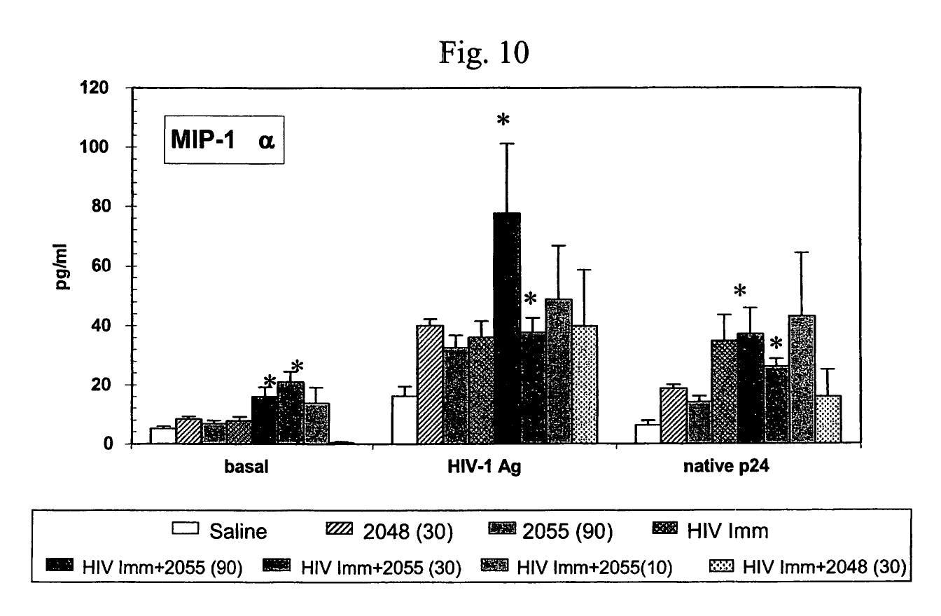

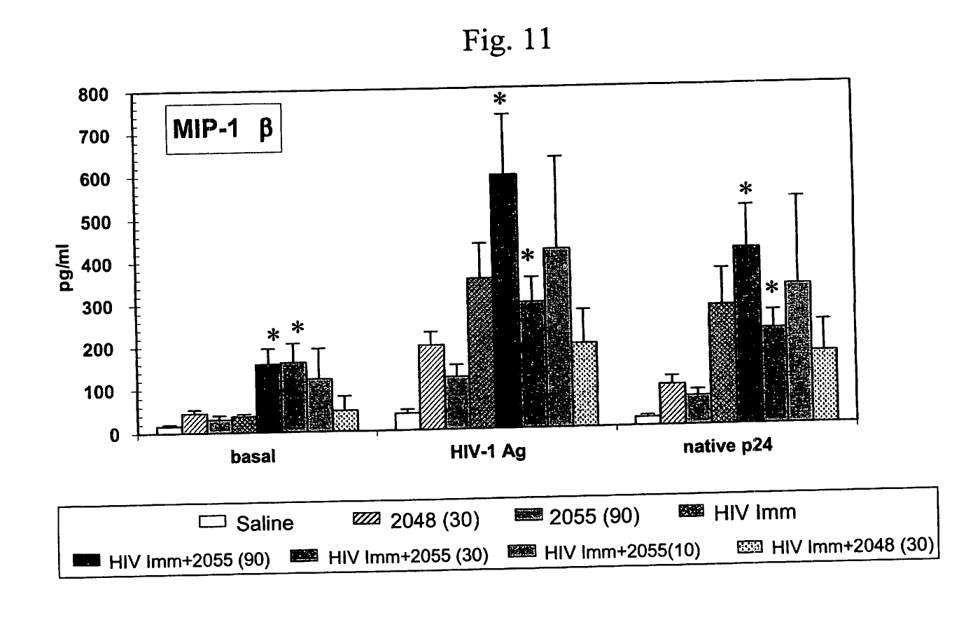

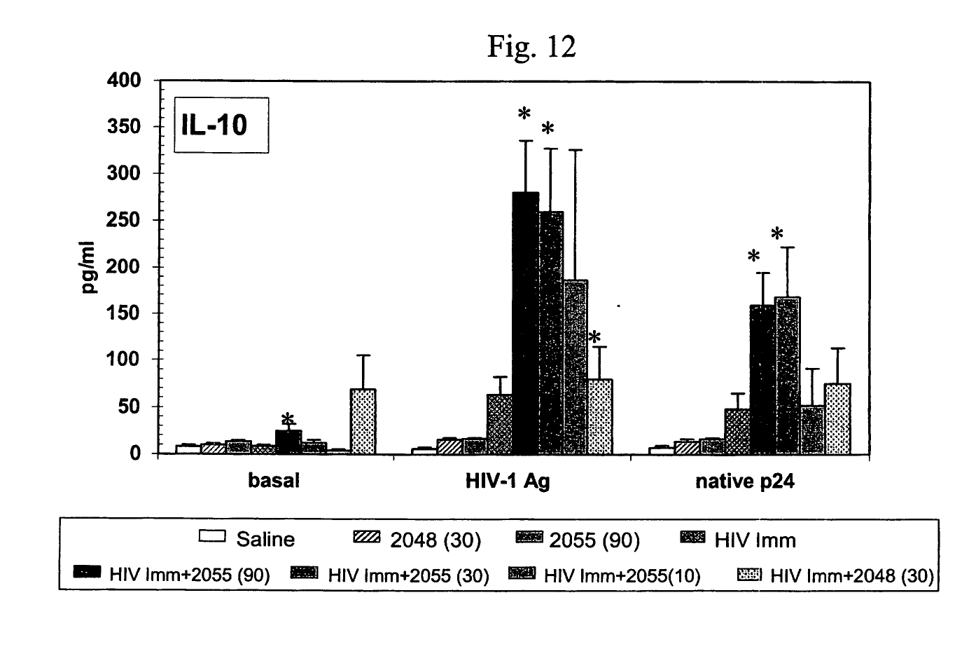

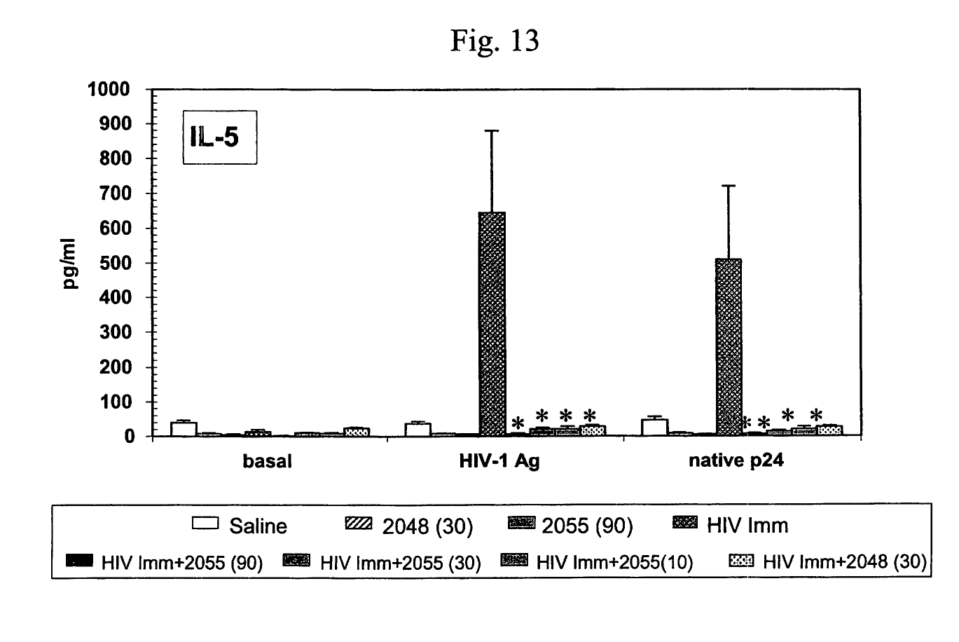

FIG. 8 shows that HIV-specific IFNγ production was enhanced by Amplivax™ in a dose dependent manner. The amount of immunomer used is shown in parentheses (μg/mouse). FIG. 9 shows that HIV-specific RANTES production was enhanced by Amplivax™ in a dose dependent manner. FIG. 10 shows that HIV-specific MIP-1α production was enhanced by Amplivax™ in a dose dependent manner. The amount of immunomer used is shown in parentheses (μg/mouse). FIG. 11 shows that HIV -specific MIP-1β production was enhanced by Amplivax™ in a dose dependent manner. The amount of immunomer used is shown in parentheses (μg/mouse). FIG. 12 shows that HIV-specific IL-10 production was enhanced by Amplivax™ in a dose dependent manner. The amount of immunomer used is shown in parentheses (μg/mouse). FIG. 13 shows that HIV-specific IL-5 production was reduced by Amplivax™ given subcutaneously. The amount of immunomer used is shown in parentheses (μg/mouse).

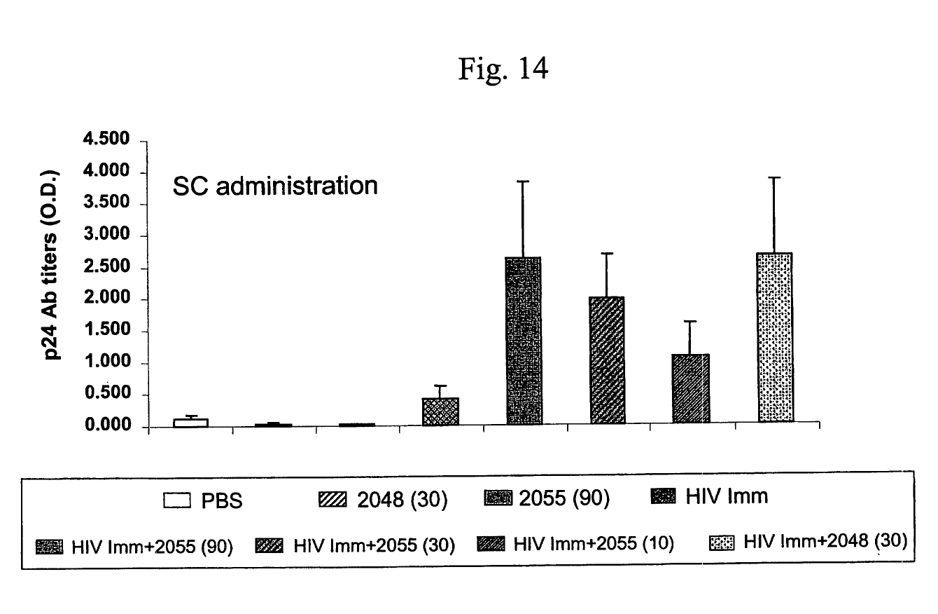

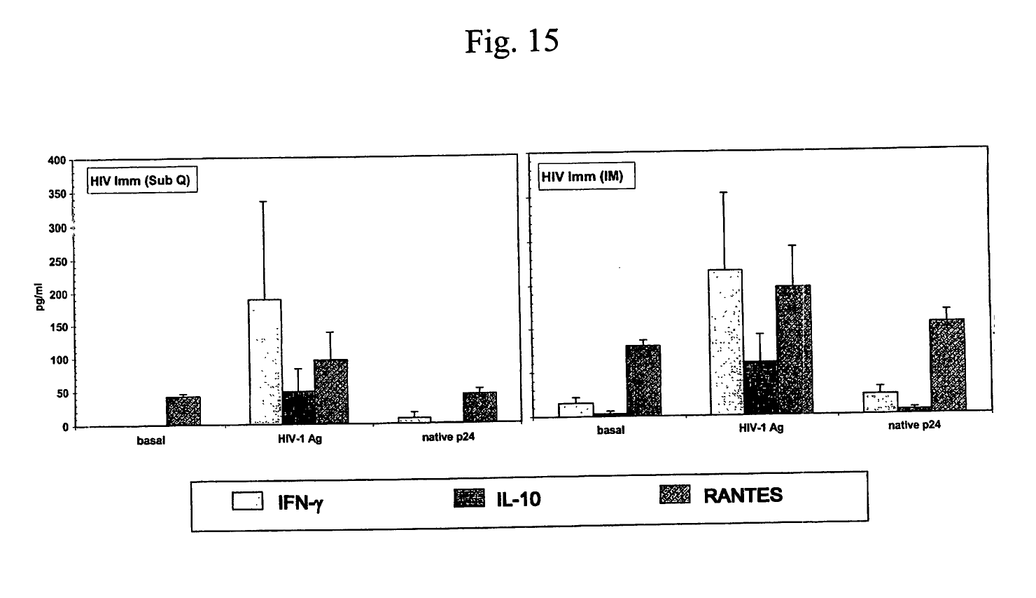

FIG. 14 shows the effect of Amplivax™ on HIV-1 immunogen-induced p24 antibody titers in mice. The amount of immunomer used is shown in parentheses (μg/mouse). FIG. 15 shows that HIV-1 whole killed vaccine in IFA (HIV-1 immunogen) induced HIV specific cytokine production upon subcutaneous (SC) and intramuscular (IM) administration.

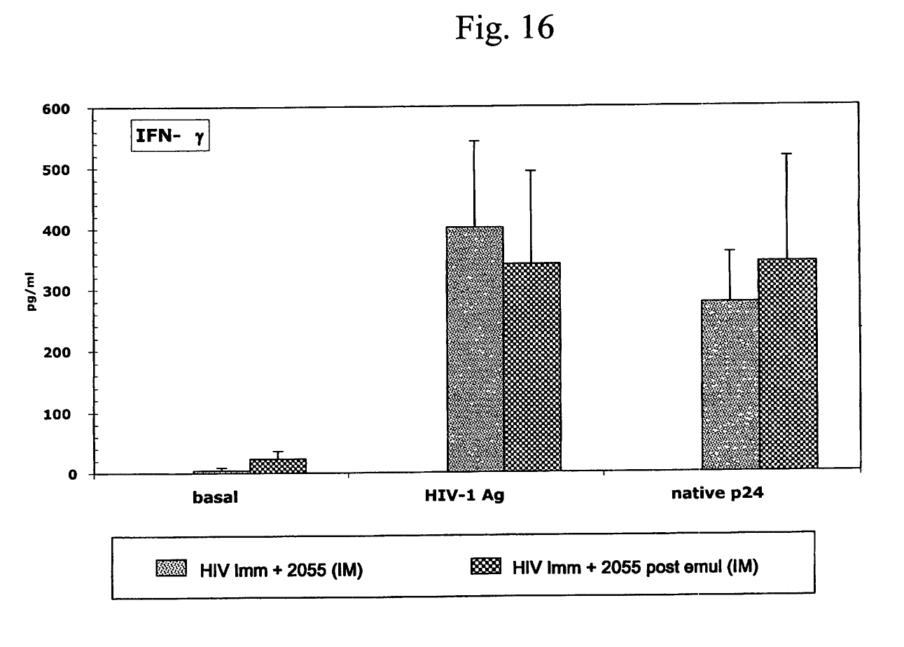

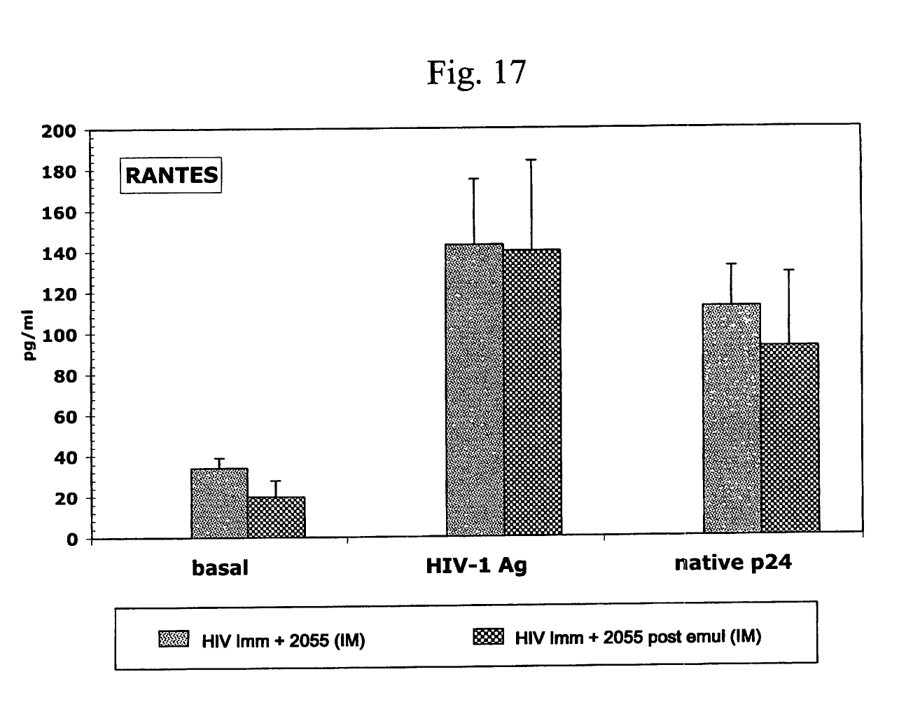

FIG. 16 shows that Amplivax™ can be added pre- or post-emulsion with IFA and enhance IFNγ production. FIG. 17 shows that Amplivax™ can be added pre- or post-emulsion with IFA and enhance RANTES production. As shown in Table 4, the combination of HIV-1 Immunogen and Amplivax™ shifts the cytokine profile toward Th1 type responses.

TABLE 4

| | IFN-γ/IL-10 | IFN-γ/IL-5 | Cytokine |
|---|---|---|---|
| | ratio | ratio | profile |
| HIV-1 Immunogen | 1.42 | 0.02 | Th2 type |
| HIV-1 Immunogen + | 74.04 | 321 | Th1 type |
| Amplivax ™ | | | |
| Amplivax ™ | — | — | — |

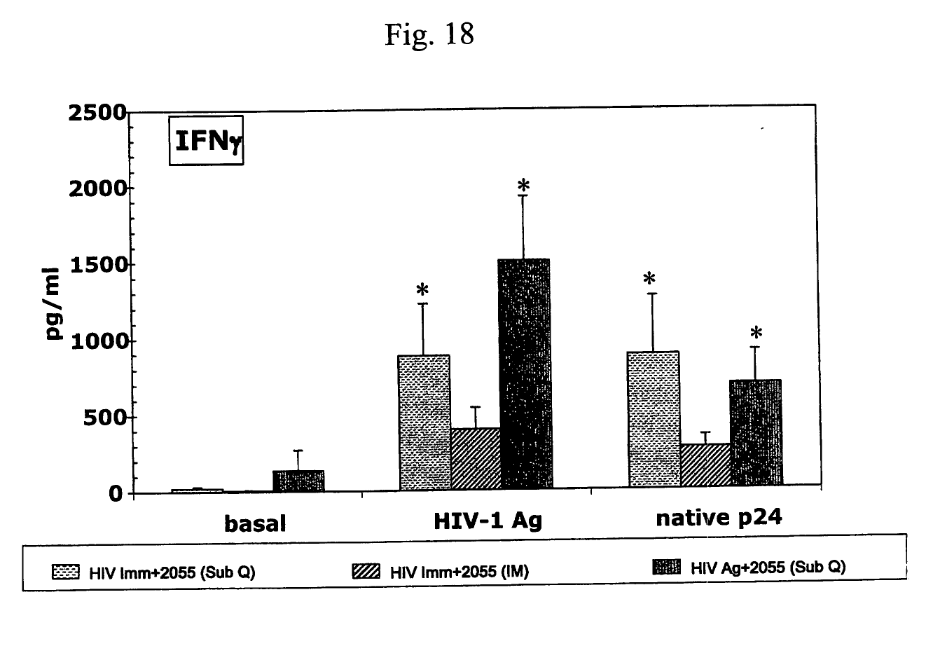

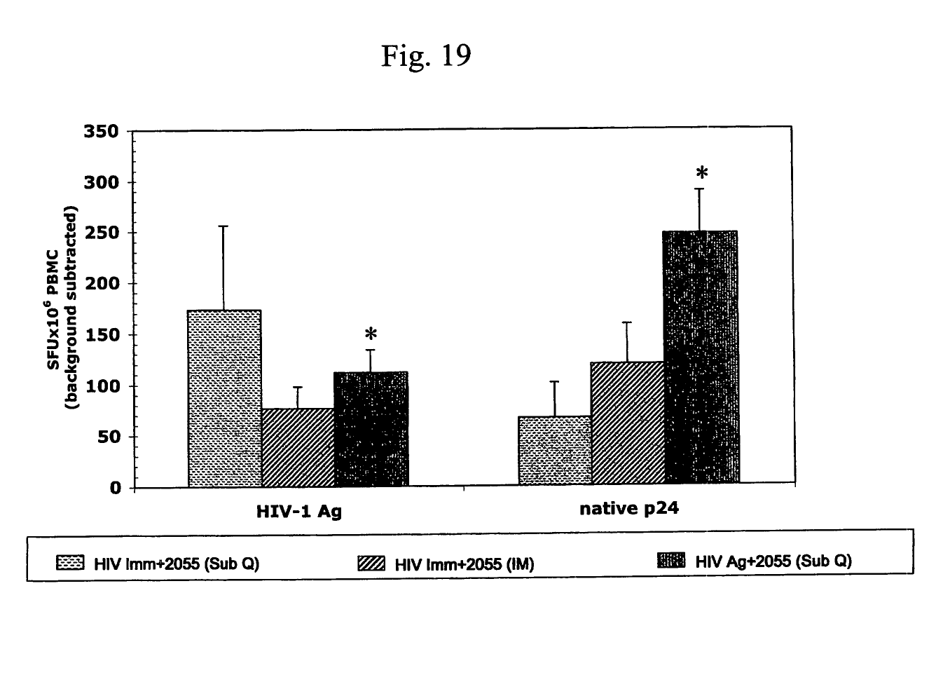

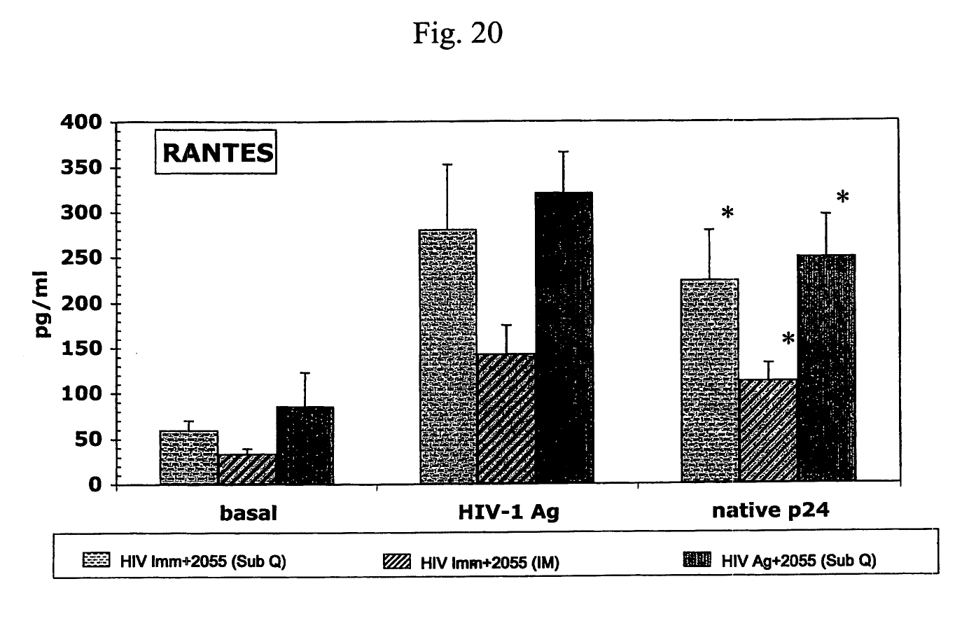

FIG. 18 shows that HIV-1 whole killed vaccine with Amplivax™ triggered HIV-specific IFNγ production in mice immunized subcutaneously without IFA. FIG. 19 shows that HIV-1 whole killed vaccine with Amplivax™ triggered HIV-specific IFNγ-secreting CD8+ T cell activity in mice immunized subcutaneously without IFA. FIG. 20 shows that HIV-1 whole killed vaccine with Amplivax™ triggered HIV-specific RANTES production in mice immunized subcutaneously without IFA.

C57/BL6 mice immunized subcutaneously with a combination of HIV-1 Immunogen and Amplivax™ showed significantly enhanced HIV-specific production of p24 antibody, HIV-specific IFN-γ (both quantity and number of CD4 and CD8 T cells producing it), chemokines (RAINTES, MIP-1α, MIP-1β), and IL-10 when compared to HIV-1 Immunogen or Amplivax™ alone. Importantly, the enhancements by Amplivax™ were still observed if HIV-1 antigen was not emulsified with IFA.

Immunization with both the combinations of Amplivax™+HIV-1 Immunogen and Amplivax™+HIV-1 Antigen significantly enhanced HIV- and p24-specific IFNγ, RANTES, MIP 1α, MIP 1β and IL-10 production as well as the number of IFNγ-producing cells compared to HIV-1 Immunogen or Amplivax alone. The magnitude of immune responses observed in mice immunized with the HIV-1 Immunogen or with the HIV-1 Antigen in combination with Amplivax was comparable.

These results show that immunization with HIV-1 Immunogen plus Amplivax™ significantly enhanced HIV-specific IFNγ, RANTES, MIP-1α, MIP-1β and IL-10 production as well as the number of IFNγ-producing cells compared to HIV-1 Immunogen or Amplivax™ alone. The magnitude of immune responses observed in mice immunized with HIV-1 antigen and Amplivax™ (without IFA) was comparable to the responses obtained with HIV-1 Immunogen (HIV-1 Antigen plus IFA) and Amplivax™. Amplivax™ in association with the whole killed virus vaccine elicits strong HIV-specific immune responses independently of the use of IFA.

Amplivax in association with either HIV-1 Immunogen or HIV-1 Antigen elicits strong virus-specific immune responses independently of the use of IFA. The strong immunogenicity of the combination of HIV-1 +Amplivax warrants its use as a therapeutic vaccine for HIV infected patients.

### EXAMPLE XI

### The Effect of HIV Immunogen in Combination With an Immunomer on In Vitro HIV-Specific Immune Responses Using Human Peripheral Blood Mononuclear Cells

This example describes the effect of Amplivax™ on in vitro HIV-specific immune responses in human peripheral blood mononuclear cells (PBMCs).

These studies were initiated to evaluate if Amplivax™ could increase in vitro HIV-specific immune responses of human PBMCs from antiretroviral-treated HIV-infected patients, who were or were not immunized with HIV-1 whole killed vaccine.

Amplivax™ was investigated ex vivo for its ability to enhance HIV antigen stimulation of PBMC isolated from HIV+patients treated with antiretroviral therapy (ART). Patients were either non-immunized, or previously immunized with HIV-1 Immunogen. Both patient groups had comparable CD4 counts, HIV plasma viremia, duration of infection, and ART. Results showed that Amplivax™ induced stronger HIV-specific cell-mediated immune responses in patients vaccinated with HIV-1 Immunogen, as measured by total spots produced in the IFN-Y ELISPOT assay and a higher percentage of alpha defensin-producing cells. Both effects were most evident using 1 μg/ml of Amplivax™.

Briefly, patients had been vaccinated with 6-24 doses of HIV whole killed vaccine (REMUNE®=HIV-1 Immunogen). The last dose was given 6-8 months before blood collection. Non-vaccinated patients were matched for CD4, HIV viremia and HAART exposure. Amplivax™ was added to PBMCs at 4 concentrations (0, 0.1, 1.0 and 10 μg/ml). Cells were stimulated with HIV-1, nP24, gag and flu antigens. The evaluation of CD8+ , IFNγ-producing cells was carried out by ELIspot. Analysis of α-defensin producing cells was by fluorescence activated cell sorting (FACS) methods.

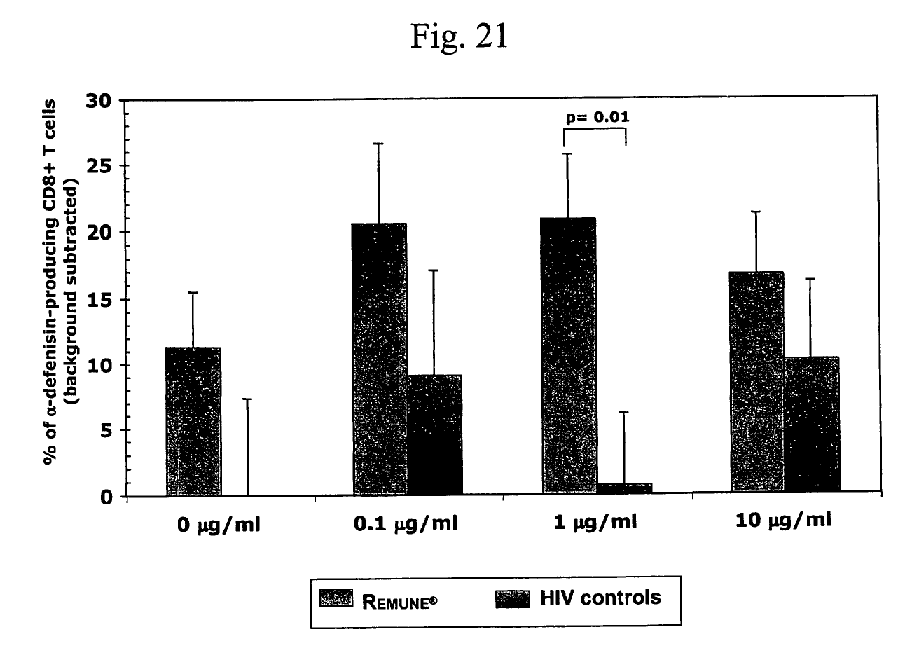

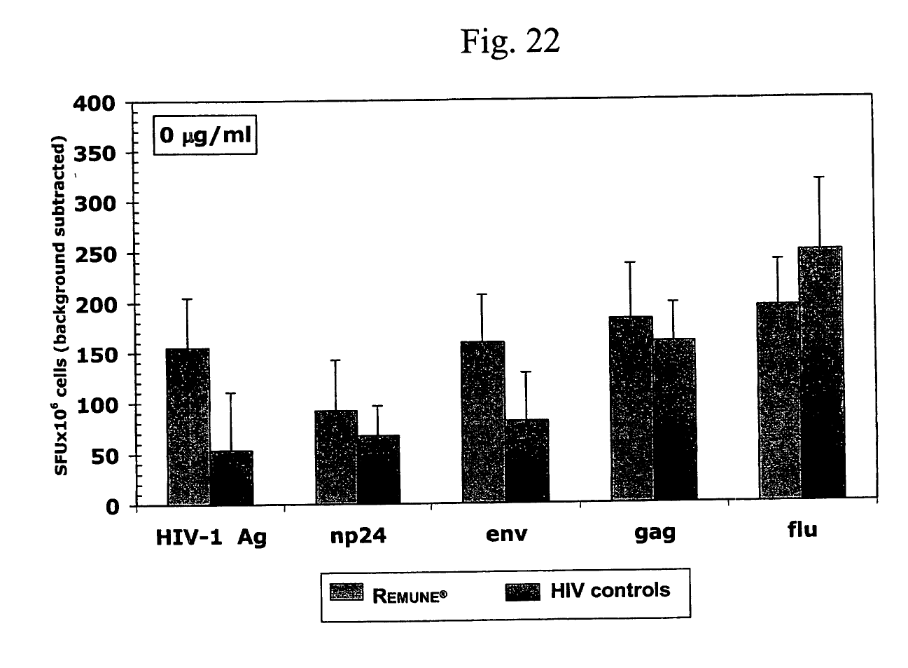

FIG. 21 shows that percentages of α-defensin producing CD8+ T cells are increased by Amplivax™ added ex vivo. FIG. 22 shows HIV-specific IFNγ-producing CD8+ T cells in REMUNE® treated patients and HIV positive controls (without

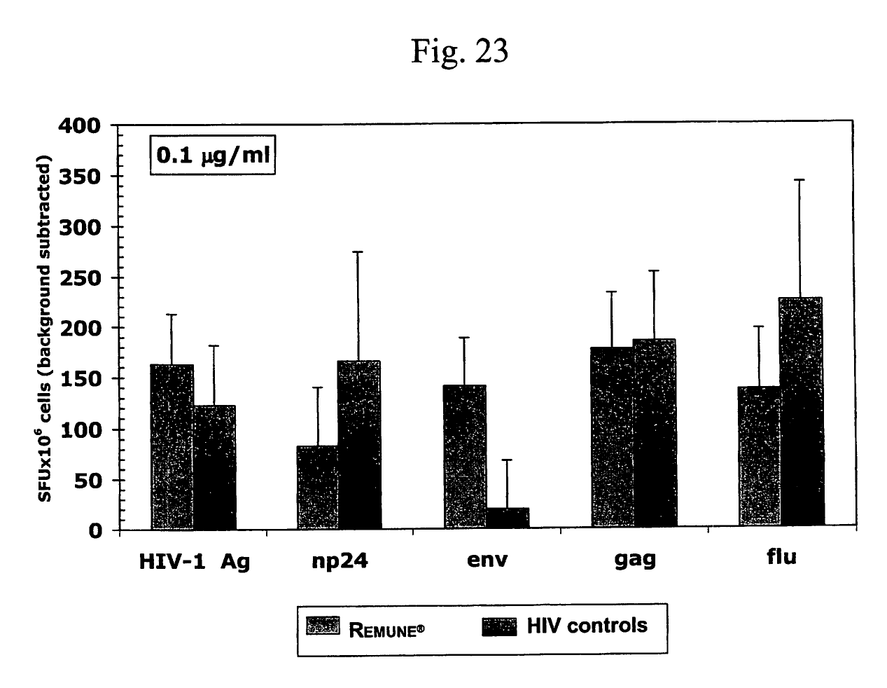

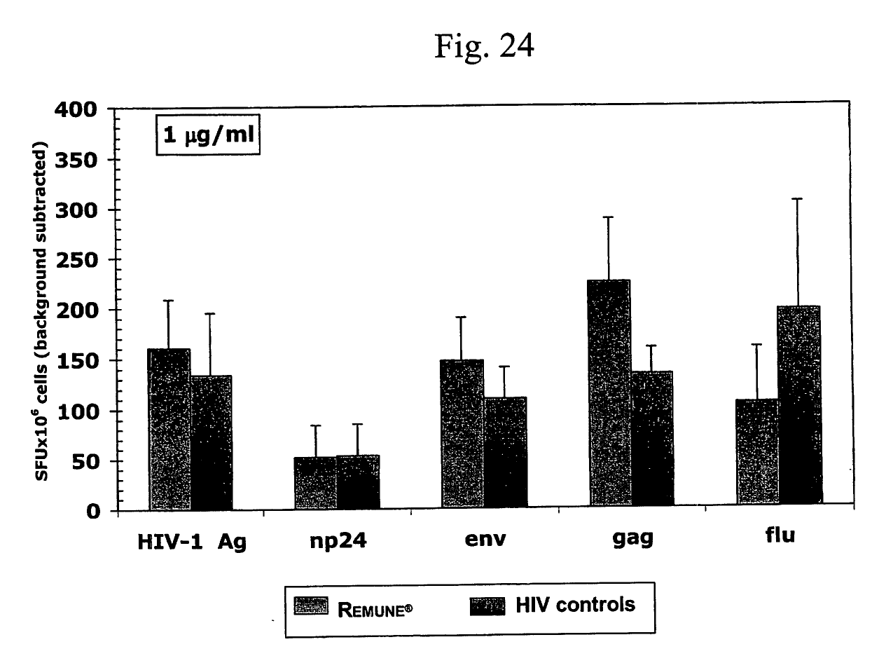

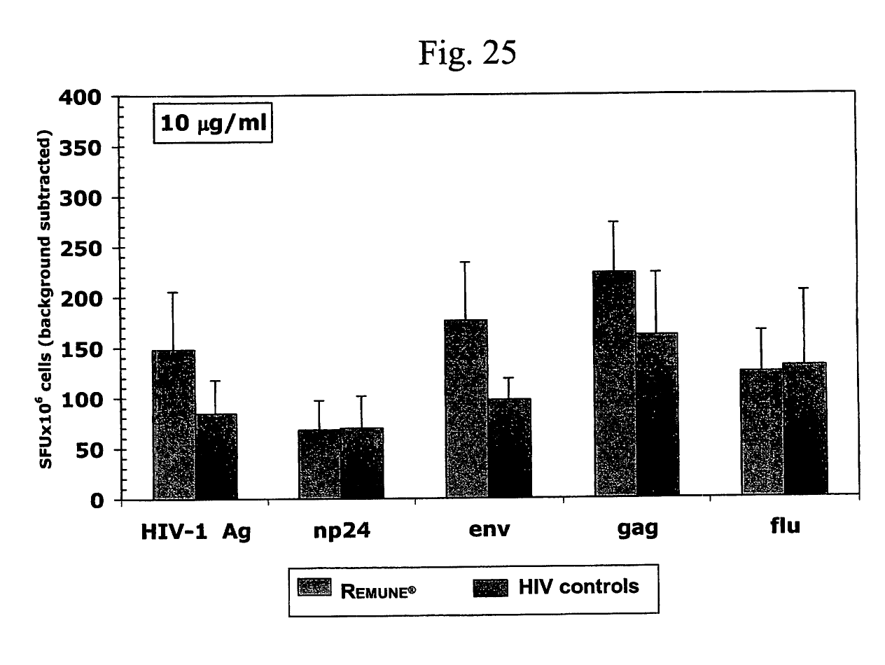

Amplivax™). FIG. 23 shows HIV-specific IFNγ-producing CD8+ T cells in the presence of 0.1 μg/ml of Amplivax™ added ex vivo. FIG. 24 shows HIV-specific IFNγ-producing CD8+ T cells in the presence of 1 μg/ml of Amplivax™ added ex vivo. FIG. 25 shows HIV-specific IFNγ-producing CD8+ T cells in the presence of 10 μg/ml of Amplivax™ added ex vivo.

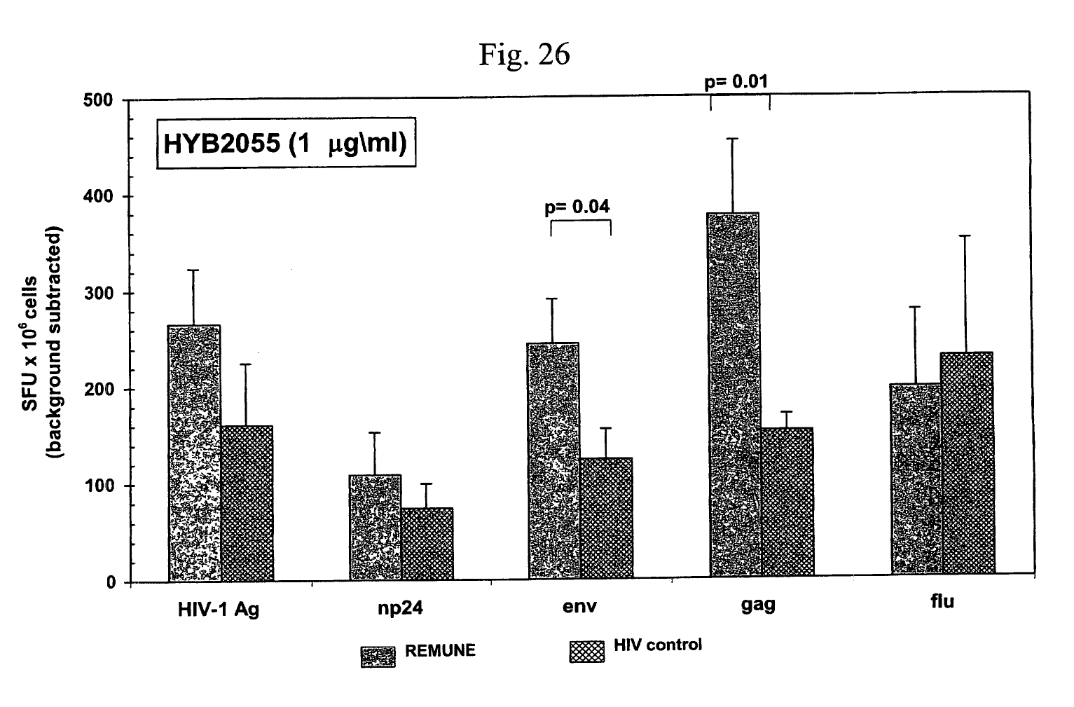

FIG. 26 shows IFN-γ ELIspot assay in peripheral blood mononuclear cells (PBMCs). HYB2055 was used at 1 μg/ml.

Preliminary data have been generated for REMUNE® (HIV-1 immunogen plus IFA) in HAART naive patients. The trial, when completed, will monitor fifty HIV-1 positive subjects with HIV-1 RNA in the range from 10,000-40,000 copies/mL and CD4 cells above 350 cells/μL. Patients were randomized into three groups: REMUNE® (HIV-1 immunogen in IFA); IFA adjuvant; or saline.

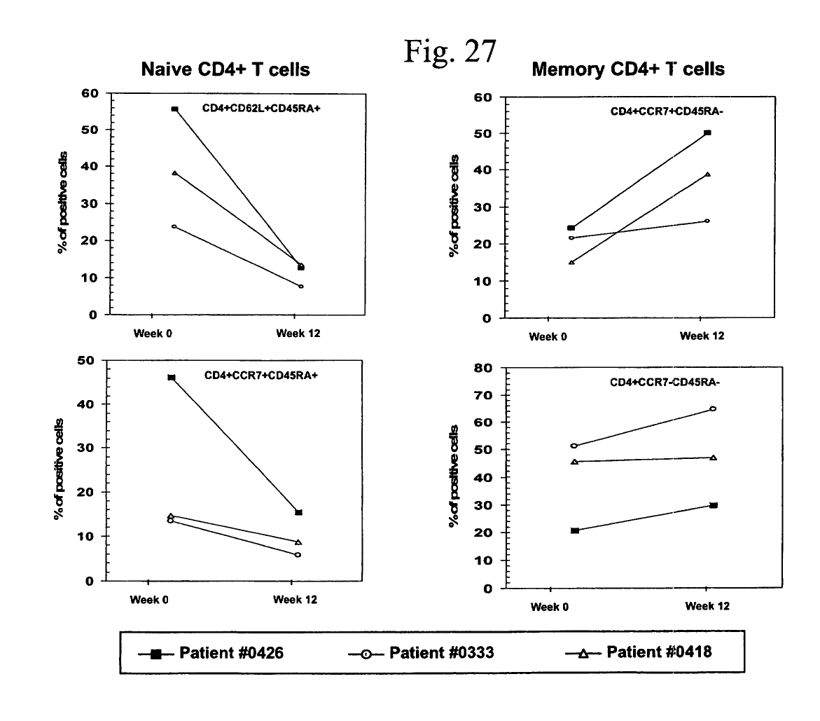

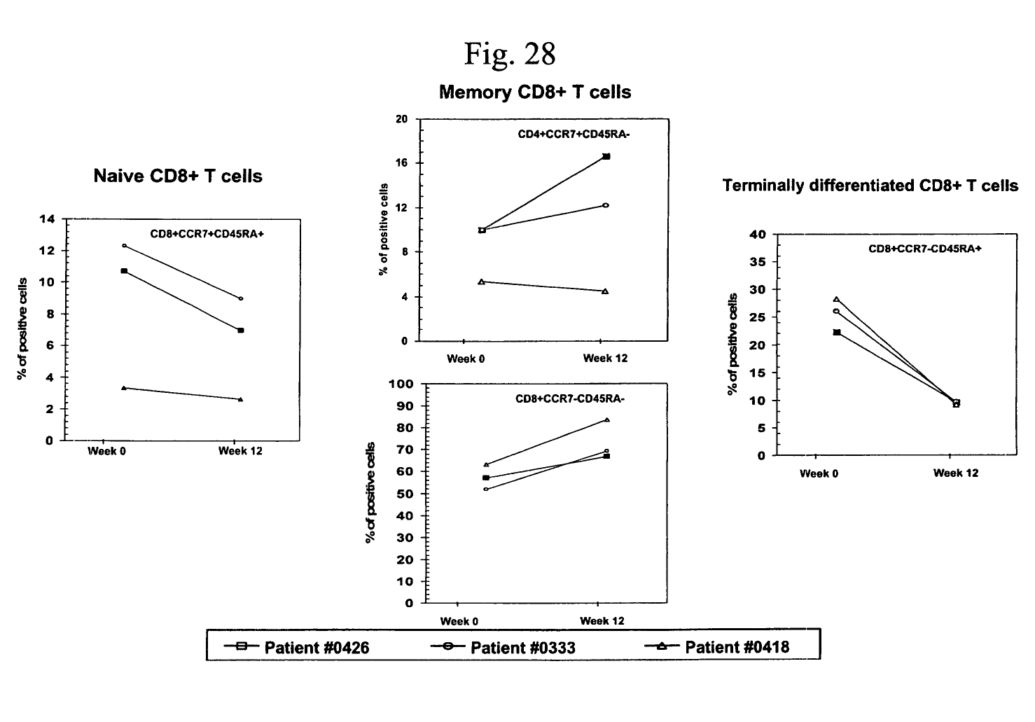

Phenotypic changes in CD4 T cells (FIG. 27) and for CD8 T cells (FIG. 28) were observed post 1st injection of REMUNES in antiretroviral therapy (ART) naive patients. Preliminary data on the first few patients are shown. Additional patients will be analyzed similarly.

Preliminary data from an ongoing clinical trial in drug-naive HIV+patients suggests that HIV-1 Immunogen also has a positive effect on generation of HIV-specific immune responses in this patient population. The potential enhancing effect of Amplivax™ will be examined in a roll over trial in these same patients by adding Amplivax™ to the HIV-1 Immunogen as part of the vaccine.

Throughout this application various publications have been referenced. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

1-34. (canceled)

35. An immunogenic composition, comprising:
(a) a whole-killed HIV virus; and
(b) an immunomer.

36. The immunogenic composition of claim 35, wherein said whole-killed HIV virus is devoid of outer envelope protein gp120.

37. The immunogenic composition of claim 35, further comprising an adjuvant.

38. The immunogenic composition of claim 35, wherein said HIV virus is HIV-1.

39. The immunogenic composition of claim 35, wherein said HIV virus is an HZ321 strain virus.

40. The immunogenic composition of claim 35, wherein said isolated nucleic acid molecule comprises a phosphorothioate backbone.

41. The immunogenic composition of claim 35, wherein said HIV virus is conjugated to said nucleic acid molecule.

42. The immunogenic composition of claim 35, wherein said immunomer is a CpG immunomer.

43. The immunogenic composition of claim 35, wherein said immunomer is a CpG-free immunomer.

44. The immunogenic composition of claim 37, wherein said adjuvant is suitable for use in humans.

45. The immunogenic composition of claim 37, wherein said adjuvant comprises incomplete Freund's adjuvant (IFA).

46. The immunogenic composition of claim 37, wherein said adjuvant comprises mycobacterium cell wall components and monophosphoryl lipid A.

47. The immunogenic composition of claim 37, wherein said adjuvant comprises alum.

48. The immunogenic composition of claim 35, wherein said composition enhances β-chemokine production.

49. The immunogenic composition of claim 35, wherein said composition enhances HIV-specific IgG2 antibody production in a mammal.

50. The immunogenic composition of claim 35, wherein said composition enhances an HIV-specific cytotoxic T lymphocyte (CTL) response in a mammal.

51. The immunogenic composition of claim 35, wherein said composition enhances HIV-specific CD4+ helper T cells.

52. The immunogenic composition of claim 35, wherein said composition induces HIV specific CD4 T helper cells and CD8+ T cells.

53. The immunogenic composition of claim 35, wherein said composition increases CD4 helper and CD8 cytotoxic T lymphocyte memory cells.

54. The immunogenic composition of claim 35, wherein said composition slows progression to AIDS in an HIV infected individual.

55. The immunogenic composition of claim 35, wherein said composition reduces viral burden in an individual infected with HIV.

56. The composition of claim 35, wherein said composition prolongs life expectancy of an individual with AIDS.

57. A kit, comprising:
a) a whole-killed HIV virus; and
b) an immunomer,
said kit components, when combined, producing the immunogenic composition of claim 35.

58. The kit of claim 57, wherein said whole-killed HIV virus is devoid of outer envelope protein gp120.

59. The kit of claim 57, further comprising an adjuvant.

60. A method of making the immunogenic composition of claim 35, comprising combining:
a) a whole-killed HIV virus; and
b) an immunomer.

61. The method of claim 60, wherein said whole-killed HIV virus is devoid of outer envelope protein gp120.

62. The method of claim 60, wherein an adjuvant is combined with said whole-killed HIV virus and said immunomer.

63. A method of immunizing a mammal, comprising enhancing an immune response in the mammal by administering to the mammal the immunogenic composition of claim 35.

64. A method of inhibiting AIDS, comprising enhancing an immune response in a mammal by administering to the mammal the immunogenic composition of claim 35.

65. The method of claim 64, wherein the asymptomatic phase of HWV infection is prolonged.

66. The method of claim 63 or 64, wherein said mammal is a primate.

67. The method of claim 66, wherein said primate is an infant.

68. The method of claim 66, wherein said primate is a child.

69. The method of claim 66, wherein said primate is pregnant.

70. The method of claim 66, wherein said primate is a human.

71. The method of claim 70, wherein said human is HIV seronegative.

72. The method of claim 70, wherein said human is HIV seropositive.

73. The method of claim 63 or 64, wherein said composition is administered to said mammal two or more times.

74. The method of claim 63 or 64, wherein the composition is administered subcutaneously, intramuscularly or intramucosally.
